(19) United States
(12) Patent Application Publication
(10) Pub. No.: US 2024/0350974 A1
(43) Pub. Date: Oct. 24, 2024

(54) SYSTEMS AND METHODS FOR PROTEIN RECOVERY

(71) Applicant: Solecta, Inc., Oceanside, CA (US)

(72) Inventors: Mariusz Grzelakowski, Temecula, CA (US); Meijuan Zhou, Oceanside, CA (US); Sanjay Srivatsa, Walnut Creek, CA (US)

(21) Appl. No.: 18/640,374

(22) Filed: Apr. 19, 2024

Related U.S. Application Data
(63) Continuation of application No. PCT/US2022/047161, filed on Oct. 19, 2022.
(60) Provisional application No. 63/257,878, filed on Oct. 20, 2021.

(51) Int. Cl. B01D 61/02; B01D 61/14; B01D 63/10; B01D 71/34; B01D 71/42; B01D 71/64; B01D 71/68; C07K 1/34

(52) U.S. Cl. CPC B01D 61/027; B01D 61/145; B01D 61/149; B01D 63/10; B01D 71/34; B01D 71/421; B01D 71/643; B01D 71/68; C07K 1/34; B01D 2325/02833; B01D 2325/14

(57) ABSTRACT

The present disclosure provides systems and methods for the recovery of protein species from wet mill grain process streams. Systems and methods of the present disclosure may be integrated with a wet mill grain process to separate out protein species that may limit efficiency of the grain process and produce one or more product streams comprising these separated protein species. A feed stream may be fractionated by at least two membranes into retentate and permeate streams. Removing larger proteins through the membrane fractionation may allow previously soluble prolamin products in the permeate stream(s) to precipitate. The recovered protein species may include prolamin, such as zein from a corn grain feed.

### CROSS-REFERENCE

This application is a continuation of International Patent Application No. PCT/US22/47161, filed Oct. 19, 2022, which claims the benefit of U.S. Provisional Application No. 63/257,878, filed Oct. 20, 2021, which application is incorporated herein by reference.

### BACKGROUND

Grain processing generally utilizes mechanical separation steps, such as filtration and gravity separations to separate the valuable human and animal food products in the grains, such as germ and gluten meal, and to produce valuable starch slurries that are used in downstream fermentation. The overflow from such a process may include desirable protein species and other compounds. Currently, these processes lose a significant portion of the valuable protein constituents contained in the grains. While these separation processes effectively capture macro-sized constituents, they are not effective in capturing fine particulates, colloidal or soluble protein species, such as lower molecular weight glutens and prolamins.

### SUMMARY

In conventional grain processing steps (e.g., corn wet mill processes), the overflow from the mechanical separations containing these fine protein particulates often find their way to the grain steep process, where they occupy unfermentable process capacity and thus limit overall grind rates. In some situations, streams containing fine particulates, colloidal or soluble species are intentionally discharged from the wet mill corn processes to avoid restricting fermentation capacity and to avoid problems in downstream processing. Types of plant materials used in grain processing methods (i.e., grain feed streams) may include, but are not limited to, corn, pea, wheat, oat, rice, wild rice, corn, barley, millet, rye, sorghum, amaranth, bulgur, farro, teff, quinoa, kamut, whole wheat pasta, couscous, sunflower, pulses, and soy materials. In current corn wet mill processes, for example, lower molecular weight glutens can be lost and practically none of the corn prolamin, zein, is recovered. Thus, there remains a need for a cost-effective approach to capture the valuable ingredients (e.g., grain proteins) contained in these fine particle, colloidal and soluble streams.

Other protein separation methods (i.e., separation of proteins from dairy streams) may use membrane separation techniques that are unsuitable for grain separation methods. For example, proteins in grain feed streams have a higher content of starch molecules (as compared to dairy feed streams. These starch molecules contribute to several challenges with membrane separation of protein species that are specific to protein separation from grain (i.e., corn, pea and soy) feed streams. Higher concentrations of starch molecules propagates increased fouling of streams through membranes. Increased fouling can also result in low or decreased flux through a system which can contribute to microbiological contamination of the feed stream. In some cases, the increased starch concentrations in grain feed streams may foul a membrane to the point of preventing commercial feasibility or causing irreversible damage to the membranes. These challenges with existing grain protein separation methods, stemming from the increased starch concentrations, can all contribute to compromised food safety. Thus, there remains a need for a protein recovery process, specifically suited to handle grain feed streams with high levels of starch molecules, which can avoid the fouling, microbiological contamination, and food safety issues with existing membrane separation methods.

Albumin is one of the pulse proteins found in the seeds of legumes such as the pea plant (Pisum sativum) or soya bean. Pulse proteins contain high levels of globulins (salt soluble proteins), which are also storage proteins. About 10-20% of pulse proteins are water-soluble albumins. Prolamins are plant storage proteins with a high proline amino acid content. Examples of prolamins include, but are not limited to, zein in corn, gliadin in wheat, hordein in barley, secalin in rye, sordhum in kafirin, and avenin in oat. They are also characterized by a high glutamine content and by poor solubility in water. Since they are soluble in alcohols, their recovery processes have been developed using alcohol extraction. However, this introduces an organic solvent which may also need be recovered, and which increases process complexity and introduces waste treatment issues. Thus, there remains a need for a protein recovery process which avoids the need for organic solvents.

Grain feed streams (i.e., corn, pea and soy feed streams) can contain a wide spectrum of target proteins that are not easily distinguishable or separatable. Methods of separating proteins from dairy streams, where such dairy proteins are distinct from other solutes in the dairy stream, have not addressed the challenge of targeting and successfully separating grain proteins. Thus, there remains a need for a protein recovery process which enables a high degree of target protein differentiation and increases target protein refinement.

Prolamin proteins, such as zein in corn, are a valuable by-product of grain processing. Taking corn wet mill processes as an example, a series of complicated steps can be required to recover zein proteins in a pure form. The current price of purified zein is $20-70 per kg depending on the grade and purity. This price limits the use of zein proteins to specialty applications that can tolerate the high price, such as pharmaceutical tablet coatings or confectionary coatings. Zein is currently an uneconomical material for large industrial uses such as biodegradable plastics. Thus, there remains a need for cost effective approach to recover zein and other prolamin proteins.

Provided herein are methods and systems for recovering valuable ingredients (e.g., proteins) from a stream (e.g., an overflow from mechanical separations) derived from a grain processing unit (e.g., a wet mill grain process). In an aspect, the methods and systems provided herein comprise a feed stream which comprises a first set of proteins and a second set of proteins. The first set of proteins may have an average molecular weight greater than about 100 kilodalton (kDa) and/or an average particle size between about 0.05 micrometers and about 0.2 micrometers. The second set of proteins may have an average molecular weight of less than about 75 kDa and/or an average particle size less than about 20 nanometers (nm). In some cases, the methods and systems provided herein comprise a first separation process comprising a first membrane, and a second separation process comprising a second membrane. The methods and systems provided herein may generate a first retentate stream, a first permeate stream, a second retentate stream and/or a second permeate stream. The first retentate stream may comprise the first set of proteins. The second retentate stream may comprise the first set of proteins. The second permeate stream may comprise the second set of proteins. The methods and systems provided herein may allow for greater than 90% recovery of prolamin from the feed stream. The methods and systems provided herein may allow for recovery of one or more protein species from one or more of the first retentate stream, first permeate stream, second retentate stream, or second permeate stream.

In an aspect, the present disclosure provides a method of recovering fine particulate, or protein species from a feed from a wet mill grain process, said method comprising: (a) subjecting the feed from the wet mill grain process to a first separation process utilizing a first membrane under conditions effective to provide a first retentate stream comprising one or more higher molecular weight proteins having an average molecular weight of greater than about 75 kilodalton and a first permeate stream comprising one or more lower molecular weight proteins having an average molecular weight of less than about 75 kilodalton; and (b) subjecting the first permeate stream to a second separation process utilizing a second membrane under conditions effective to provide a second retentate stream comprising glutens and a second permeate stream comprising prolamins.

In some embodiments, the first membrane comprises a microporous membrane.

In some embodiments, the first membrane comprises an ultrafiltration membrane.

In some embodiments, the second membrane comprises a nanofiltration membrane.

In some embodiments, the one or more lower molecular weight proteins comprise glutens or prolamins.

In some embodiments, the one or more lower molecular weight proteins comprise glutens and prolamins.

In some embodiments, the method further comprises an additional separation process utilizing at least one spiral wound membrane element.

In some embodiments, the method further comprises an additional separation process utilizing at least two spiral wound membrane elements in series.

In some embodiments, the at least two spiral wound membrane elements are in parallel.

In some embodiments, the one or more higher molecular weight proteins comprise glutens.

In some embodiments, the method further comprises directing the second permeate stream to pass through a nanofiltration membrane unit to recover protein species comprising prolamins.

In some embodiments, an ultrafiltration step precedes the nanofiltration step of said nanofiltration membrane unit.

In some embodiments, recovery of a stream comprising prolamins is increased by removal of a stream comprising glutens.

In some embodiments, the method further comprises recovering one or more protein species or non-protein species from at least one of (i) the first retentate stream, (ii) the first permeate stream, (iii) the second retentate stream, and (iv) the second permeate stream.

In some embodiments, the recovered protein species comprise glutelins, prolamins or glutens.

In some embodiments, the recovered non-protein species comprise carbohydrates, starches, enzymes, alcohols, aldehydes, fats or other low molecular weight organic species.

In some embodiments, a grain feed to the wet mill grain process comprises corn, wheat, barley, rye, oats, or rice.

In some embodiments, the feed from the wet mill grain process to the first separation process is supplied from an overflow of a mechanical separation device or gravity separation device.

In some embodiments, the feed from the wet mill grain process is an aqueous stream with no added organic solvent.

In some embodiments, the first membrane comprises a low fouling spiral wound membrane comprising at least one polysulfone, polyvinyl difluoride, polyethersulfone, polyacrylonitrile, or polyetherimide.

In some embodiments, the first membrane material comprises polysulfone, polyvinyl difluoride, or polyethersulfone.

In some embodiments, the second membrane comprises a nanofiltration membrane which is a low fouling spiral wound membrane comprising at least one polysulfone, polyvinyl difluoride, polyethersulfone, polyacrylonitriles, or polyetherimide material as a microporous substrate and comprises a top interfacial coating or separation layer.

In some embodiments, the second membrane comprises a nanofiltration membrane material comprising a microporous polysulfone membrane as substrate and a top interfacial crosslinked polyamide layer.

In some embodiments, the spiral wound membrane elements include a spacer element with a thickness equal to or between about 30 mils and about 270 mils thickness.

In some embodiments, the spiral wound membrane elements include a spacer element with a thickness equal to or between about 30 mils and about 135 mils thickness.

In some embodiments, the first separation process or the second separation process is operated at a feed pressure of about 15 pounds per square inch guage (psig) to about 200 psig.

In some embodiments, a nominal pore size of the first membrane is between about 0.02 microns and about 0.50 microns.

In some embodiments, a nominal pore size of the first membrane is between about 0.05 and about 0.20 microns.

In some embodiments, the first retentate stream comprises at least about 90% of the insoluble particulates greater than about 0.5 microns in diameter.

In some embodiments, a reject stream from the first membrane comprises about 5% to about 70% of high molecular weight solubles and fine particulates greater than about 0.5 microns (or micrometer) in size.

In some embodiments, the second membrane is a nanofiltration membrane.

In some embodiments, a nominal pore size of the nanofiltration membrane is less than about 20 nanometers.

In some embodiments, the nanofiltration membrane is a thin composite membrane.

In some embodiments, the thin film composite nanofiltration membrane comprises a microporous membrane substrate with a pore size in between about 0.05 microns and about 0.2 microns.

In some embodiments, thin film composite nanofiltration membrane comprises a top nanoporous polymer layer of pore size less than about 20 nanometers, or with a molecular weight cut-off less than about 5000.

In some embodiments, the thin film composite nanofiltration membrane comprises a top nanoporous polymer layer produced by a dip-coating or interfacial polymerization process.

In some embodiments, the nanofiltration membrane is formed directly using a phase inversion process.

In some embodiments, the nanofiltration membrane removes at least about 80% of the low molecular weight solubles in the first permeate stream.

In some embodiments, a temperature of the overflow stream and the first or second separation process is between about 25° C. and about 65° C.

In some embodiments, a recovery stream comprises greater than about 10% of low molecular weight proteins as originally comprised in the feed from the wet mill grain process.

In some embodiments, a recovery stream comprises about 1 to about 99% of the prolamin contained in the feed from the wet mill grain process.

In some embodiments, the first microporous membrane has a molecular weight cut-off (MWCO) in the range of about 2,000 to about 5,000.

In some embodiments, the first microporous membrane has a MWCO less than about 5,000.

In some embodiments, the second microporous membrane has a MWCO in the range of about 2,000 to about 5,000.

In some embodiments, the second microporous membrane has a molecular weight cut off in the range of about 2,000 to about 5,000.

In some embodiments, one or both of said first membrane and said second membrane comprises a charged membrane. In some embodiments, said charged membrane is characterized by a net negative charge. In some embodiments, said charged membrane comprises one or more functional groups along a surface of said charged membrane. In some embodiments, said one or more functional groups comprises one or more charged chemical groups or polarizable chemical groups, and wherein a membrane charge of said membrane, or said one or more functional groups, is tunable.

In another aspect, the present disclosure provides a process integrated with a wet mill grain preprocess, comprising (a) receiving from the wet mill grain preprocess a stream as a feed to one or more separation processes which comprise at least an ultrafiltration process and a nanofiltration process; (b) subjecting said feed to said ultrafiltration process utilizing a microporous membrane to generate a first retentate stream comprising higher molecular weight proteins comprising glutens and a first permeate stream comprising prolamins, wherein the higher molecular weight proteins have an average molecular weight of greater than about 75 kilodalton (kDa); (c) directing the first permeate stream to the nanofiltration process to generate a second retentate stream comprising lower molecular weight proteins comprising glutens and a second permeate stream comprising the prolamins, wherein the lower molecular weight proteins have an average molecular weight of less than about 75 kDa; (d) recovering the prolamins from the second permeate stream; and (e) returning at least a portion of the second permeate stream to the wet mill grain preprocess.

In some embodiments, the process further comprises returning the first retentate stream to the wet mill grain preprocess or recovering from the first retentate stream the glutens.

In some embodiments, recovering the glutens comprises the use of a separation device and returning a liquid stream from the separation device to the wet mill grain preprocess.

In some embodiments, the process further comprises returning the second retentate stream to the wet mill grain preprocess or recovering from the second retentate stream the glutens.

In some embodiments, recovering the glutens from the second retentate stream comprises the use of a separation device and returning a liquid stream from the separation device to the wet mill grain preprocess.

In some embodiments, the recovering of (d) comprises the use of a separation device comprising a spray dryer, decanter, centrifuge, filter, membrane element, or any combination thereof.

In some embodiments, the stream is from an overflow from a mechanical separation device.

In some embodiments, the nanofiltration process utilizes a nanofiltration membrane.

In some embodiments, one or both of said first membrane and said second membrane comprises a charged membrane. In some embodiments, said charged membrane is characterized by a net negative charge. In some embodiments, said charged membrane comprises one or more functional groups along a surface of said charged membrane. In some embodiments, said one or more functional groups comprises one or more charged chemical groups or polarizable chemical groups, and wherein a membrane charge of said membrane, or said one or more functional groups, is tunable.

In another aspect, the present disclosure provides a process integrated with a wet mill grain preprocess, comprising: (a) receiving from the wet mill grain preprocess a stream as a feed one or more filtration processes comprising at least a first membrane filtration process and a second membrane filtration process; (b) subjecting the feed to the first membrane filtration process utilizing a microporous membrane to generate a first retentate stream comprising higher molecular weight glutens having an average molecular weight of at least about 75 kDa and a first permeate stream comprising prolamins; (c) directing the first permeate stream to the second membrane filtration process to generate a second retentate stream comprising lower molecular weight glutens having an average molecular weight of less than about 75 kDa and a second permeate stream comprising the prolamins; (d) recovering the prolamins from the second permeate stream; and (e) returning at least a portion of the second permeate stream to the wet mill grain preprocess.

In some embodiments, the process further comprises returning the first retentate stream to the wet mill grain preprocess or recovering from the first retentate stream the higher molecular weight glutens.

In some embodiments, recovering the higher molecular weight glutens comprises the use of a separation device and returning a liquid stream from the separation device to the wet mill grain preprocess.

In some embodiments, the process further comprises returning the second retentate stream to the wet mill grain preprocess or recovering from the second retentate stream the lower molecular weight glutens.

In some embodiments, recovering the lower molecular weight glutens from the second retentate stream comprises the use of a separation device and returning a liquid stream from the separation device to the wet mill grain preprocess.

In some embodiments, the recovering of (d) comprises the use of a separation device comprising a spray dryer, decanter, centrifuge, filter, membrane element, or any combination thereof.

In some embodiments, the stream is from an overflow stream from a mechanical separation device.

In some embodiments, the wet mill grain preprocess is a corn, wheat, barley, rye, sorghum or oat wet mill preprocess.

In some embodiments, the wet mill grain preprocess is a steep process.

In some embodiments, overflow stream from the mechanical separation device in the wet mill grain preprocess is an overflow from a thickener, decanter or centrifuge or a filtrate from a filter.

In some embodiments, the total solids contained in an overhead stream from the wet mill grain preprocess contains less than about 5% solids.

In some embodiments, the stream from the wet mill grain preprocess is in fluid communication with the microporous membrane utilized in the first membrane filtration process and the first retentate stream is in fluid communication with the wet mill grain preprocess or at least one separation device.

In some embodiments, a liquid stream from the at least one separation device is in fluid communication with the wet mill grain preprocess.

In some embodiments, the first permeate stream from the first membrane filtration process is in fluid communication with a nanofiltration membrane utilized in the second membrane filtration process.

In some embodiments, the second retentate stream from the nanofiltration membrane is in fluid communication with the wet mill grain process or at least one separation device.

In some embodiments, a liquid stream from the at least one separation device in the second membrane filtration process is in fluid communication with the wet grain preprocess.

In some embodiments, recovery of a stream comprising prolamins is increased by removal of a stream comprising glutens.

In some embodiments, the process further comprises recovering one or more protein species or non-protein species from at least one of (i) the first retentate stream, (ii) the first permeate stream, (iii) the second retentate stream, and (iv) the second permeate stream.

In some embodiments, the recovered protein species comprise glutelins, prolamins, or glutens.

In some embodiments, the recovered non-protein species comprise carbohydrates, starches, enzymes, alcohols, aldehydes, fats, or other low molecular weight organic species.

In some embodiments, the second retentate stream and the second permeate stream comprising the glutens and prolamins from the second membrane filtration process are recovered by centrifugation, spray-drying, decanting, filtration, or an additional membrane step.

In some embodiments, the recovered prolamins are further purified to provide high molecular weight prolamin products having an average molecular weight of between about 10 kDa and about 50 kDa.

In some embodiments, the microporous membrane is a low fouling spiral wound membrane comprising at least one polysulfone, polyethersulfone, or polyvinyl difluoride membrane element.

In some embodiments, nominal pore size range for the microporous membrane is equal to or between about 0.1 micron and about 2 microns.

In some embodiments, a nominal pore size range for the microporous membrane is equal to or between about 0.05 micron and about 0.20 microns.

In some embodiments, the first membrane filtration process or the second membrane filtration process comprises elements having spacers of equal to or between about 30 mils and about 270 mils thickness.

In some embodiments, the first retentate stream comprises at least about 90% of insoluble particulates greater than about 0.5 micron in diameter.

In some embodiments, a reject stream from the microporous membrane comprises equal to or between about 5.0% and about 70% of the high molecular weight solubles and fine particulates larger than about 0.5 microns in diameter.

In some embodiments, the second membrane filtration process utilizes a nanofiltration membrane which is a low fouling spiral wound membrane comprised of at least one of polysulfone, polyvinyl difluoride, polyethersulfone, polyacrylonitrile, and polyetherimide.

In some embodiments, the nanofiltration membrane is a low fouling spiral wound membrane comprised of at least one of polysulfone or polyvinyl difluoride.

In some embodiments, a nominal pore size of the nanofiltration membrane is less than or equal to about 10 nm.

In some embodiments, a nominal pore size of the nanofiltration membrane is less than or equal to about 5 nm.

In some embodiments, the nanofiltration membrane is a thin film composite membrane.

In some embodiments, the thin film composite membrane comprises a microporous membrane substrate with pore size in the range of about 0.05 micron to about 0.2 microns.

In some embodiments, the thin film composite membrane comprises a top nanoporous polymer layer of pore size less than about 20 nm or with a molecular weight cut-off less than about 5000.

In some embodiments, the thin film composite membrane comprises a top nanoporous polymer layer produced by a dip-coating or interfacial polymerization process.

In some embodiments, the thin film composite membrane is formed directly using a phase inversion process.

In some embodiments, the nanofiltration membrane removes at least about 80% of the low molecular weight solubles in the first permeate stream.

In some embodiments, base materials selected for the microporous and nanofiltration membranes are the same or different.

In some embodiments, a temperature of the overflow stream from the wet mill grain preprocess and the one or more filtration processes are in the range of about 25 to about 65° C.

In some embodiments, the prolamin recovered is equal to or between about 1 and about 99% of prolamin contained in the feed.

In another aspect, the present disclosure provides a membrane protein recovery system integrated with a wet mill grain system, the membrane protein recovery system comprising: (a) at least one gluten starch separator; (b) at least one gluten thickener; (c) at least one grain steeping vessel; and (d) at least two spiral wound membranes; wherein at least one of the at least two spiral wound membranes is in fluid contact with the at least one gluten thickener; and wherein at least one of the at least two spiral wound membranes or the at least one gluten starch separator is in fluid contact with the at least one grain steeping vessel.

In some embodiments, the system further comprises at least one microporous membrane in fluid contact with the at least one gluten thickener.

In some embodiments, the system further comprises a separation device in fluid communication with the at least one gluten thickener.

In some embodiments, the system further comprises a separation device in fluid contact with the at least one grain steeping vessel.

In some embodiments, the system further comprises a spray-drying device in fluid contact with the at least one grain steeping vessel, or at least one of the at least two spiral wound membranes.

In another aspect, the present disclosure provides a method for processing a feed stream, comprising: (a) providing said feed stream comprising a first set of proteins having an average molecular weight of greater than about 75 kDa and a second set of proteins having an average molecular weight of less than or equal to about 75 kDa, which second set of proteins comprises a gluten and a prolamin; (b) bringing said feed stream in contact with a first membrane under conditions sufficient to provide (i) a first retentate stream comprising said first set of proteins and (ii) a first permeate stream comprising said second set of proteins; and (c) bringing said first permeate stream in contact with a second membrane under conditions sufficient to provide a second retentate stream comprising said gluten and a second permeate stream comprising said prolamin.

In some embodiments, the first membrane comprises pores having a first average pore size and the second membrane comprises pores having a second average pore size, where the first average pore size is greater than said second average pore size.

In some embodiments, the feed stream is generated from a wet mill grain process.

In some embodiments, a grain feed of said wet mill grain process comprises corn, wheat, barley, rye, oats, or rice.

In some embodiments, the feed stream is supplied from a mechanical separation device or a gravity separation device.

In some embodiments, the first membrane is a spiral wound membrane.

In some embodiments, the second membrane is a spiral wound membrane.

In some embodiments, the feed stream is an aqueous stream with no added organic solvent.

In some embodiments, the first membrane comprises one or more of polysulfone, polyvinyl difluoride, polyethersulfone, polyacrylonitrile, and polyetherimide.

In some embodiments, the second membrane comprises one or more of polysulfone, polyvinyl difluoride, polyethersulfone, polyacrylonitrile, and polyetherimide.

In some embodiments, the second membrane comprises a microporous membrane substrate and a top interfacial crosslinked polyamide layer.

In some embodiments, the first set of proteins has an average molecular weight of greater than about 100 kDa.

In some embodiments, the first set of proteins has an average molecular weight of between about 75 kDa and about 100 kDa.

In some embodiments, the first membrane is a spiral wound membrane comprising at least one spacer element with a thickness between about 30 mils and about 135 mils.

In some embodiments, the first average pore size is between about 0.02 micrometers and about 0.5 micrometers.

In some embodiments, the second average pore size is less than about 50 micrometers.

In some embodiments, the second average pore size is less than about 20 micrometers.

In some embodiments, the first membrane has an average molecular weight cut off of between about 2 kDa and about 500 kDa.

In some embodiments, the first membrane has an average molecular weight cut off of between about 100 kDa and about 200 kDa.

In some embodiments, the feed stream further comprises fine particulates, and said first retentate stream comprises between about 5% to about 70% of said fine particulates greater than about 0.5 micrometers in diameter.

In some embodiments, the feed stream comprises insoluble particulates and said first retentate stream comprises at least about 90% of said insoluble particulates greater than about 0.5 micrometers in diameter.

In some embodiments, one or both of said first membrane and said second membrane comprises a charged membrane. In some embodiments, said charged membrane is characterized by a net negative charge. In some embodiments, said charged membrane comprises one or more functional groups along a surface of said charged membrane. In some embodiments, said one or more functional groups comprises one or more charged chemical groups or polarizable chemical groups, and wherein a membrane charge of said membrane, or said one or more functional groups, is tunable.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 provides a generalized process flow diagram of a wet mill grain process, showing recovery of germ, gluten meal and starch.

FIG. 2 is a schematic of an example two-step membrane protein recovery process integrated with a wet mill grain process, at the gluten thickener overflow (MST).

FIG. 3 is a schematic showing an example method by which a stream from mechanical separation device, rotary vacuum filter (RVF), in a wet mill grain process is in fluid communication with a fluid channel of a two-stage membrane protein recovery process.

FIG. 4 is a schematic showing a zein recovery process of the prior art using alcohol extraction.

FIG. 5 shows the precipitated prolamin after nanofiltration and dilution.

FIG. 6 shows the presence of prolamin within the precipitated white powder of FIG. 5.

FIG. 7 shows the feed from the thickener, as a gluten source, compared to the permeate from subjecting the feed to a first nanofiltration membrane.

FIG. 8 is a stained gel of pea proteins.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least” or “greater than” applies to each one of the numerical values in that series of numerical values.

Whenever the term “no more than,” “less than,” or “less than or equal to” precedes the first numerical value in a series of two or more numerical values, the term “no more than” or “less than” applies to each one of the numerical values in that series of numerical values.

The term “about” or “nearly” as used herein generally refers to within (plus or minus) 15%, 10%, 9%, 8%, 7%, 6%, 5%, 4%, 3%, 2%, or 1% of a designated value.

As used herein, the singular forms “a”, “an”, and “the” include plural references unless the context clearly dictates otherwise.

### Protein Separations

In an aspect, the present disclosure provides methods and systems which facilitate recovery of valuable ingredients (i.e., target proteins) from a grain process (e.g., a wet mill grain process of corn, pea, or soy material). These methods and systems may utilize several components that enable higher levels of protein separation, purity of final products, lower fouling, lower microbiological contamination, increased food safety, increased longevity of membranes used in the system, and other advantages.

The components of such a method or system may include, but are not limited to, membrane design, operating condition or parameters, and overall system configuration.

In some embodiments, the valuable ingredients comprise proteins in plants. In some embodiments, the plants comprise grains. In some embodiments, the grains include, but are not limited to, corn, pea, soy, wheat, oat, rice, wild rice, corn, barley, millet, rye, sorghum, amaranth, bulgur, farro, teff, quinoa, kamut, whole wheat pasta, and couscous. In some embodiments, the proteins are plant storage proteins. In some embodiments, the plant storage proteins comprise albumin, globulin, prolamin, and glutelin. Non-limiting examples of the prolamin include zein in corn, gliadin in wheat, hordein in barley, secalin in rye, sordhum in kafirin, and avenin in oat. In some embodiments, the proteins are pea proteins. Non-limiting pea proteins include, but are not limited to, glycinin, albumin, legumin, vicilin, glycinin, and β-conglycinin. In some embodiments, the proteins may be positively charged at a certain pH. In some embodiments, the proteins may be negatively charged at a certain pH. In some embodiments, charge of the proteins may be neutral at a certain pH.

### Membrane Design

The hydrophobic surface of polystyrene (PS), polyethersulfone (PES), and polyacrylonitrile (PAN) membranes may induce fouling, which is caused by hydrophobic interactions of the membrane surface among other things with proteins or other biomolecules from the mixture to be filtered. This leads to irreversible adsorption, denaturation, and aggregation of the proteins on the membrane surface and thus clogging of the membrane pores, whereby the throughput capacity of the membrane decreases sharply. In addition to the fouling, the poor water wettability of the hydrophobic polymers is disadvantageous since all of the filtration applications are based on aqueous systems.

Modification of the membrane comprises maintaining the stability of the base polymer and generating hydrophilic groups on the surface. For this purpose, examples may be divided into three categories: copolymerization/graft polymerization of a hydrophilic monomer onto a hydrophobic membrane, production of a coating or a blend of a hydrophilic polymer with the membrane polymer, and chemical modification of the membrane polymer.

Methods for the surface modification comprise grafting of PES, polysulfone (PSf), or PAN with hydrophilic acrylates, methacrylates, or acrylamides in the presence of a crosslinker (e.g., tetraethylene glycol diacrylate or N,N′-methylenebisacrylamide). The coatings are produced either by thermally-initiated polymerization in the presence of an initiator [eg, 4,4′-azobis (4-cyanovaleric acid)], by UV-initiated photoinitiator polymerization (eg, ammonium persulfate), or by electron or y irradiation. Following the reaction, the membrane may be purged in boiling methanol to wash out the remainder of the partially toxic monomers and initiators. A disadvantage of these methods is the use of toxic and sensitizing monomers, solvents, and initiators. This includes intensive cleaning steps with highly flammable solvents such as poisonous methanol. In addition, such acrylate modifications are not stable in the basic medium, but this is necessary for a purification of the membrane. Furthermore, the coating causes a reduction of the pores and thus a deterioration of the throughput compared to the unmodified membrane.

The preparation of a hydrophilic membrane may be described by preparing a blend (ie, a blend) of PES, PSf or polyvinylidene fluoride (PVDF) with hydrophilic polymers such as polyvinylpyrrolidone. The blend was crosslinked after preparation of the membrane in the case of the presence of double bonds in addition by thermally initiated polymerization with the addition of the initiator ammonium persulfate or alternatively by y-irradiation or electron beam. However, such a blend is not permanently stable and polyvinylpyrrolidone is gradually washed out during the use of the membrane. This leads to a deterioration of the membrane properties and contamination of the permeate, which is highly undesirable, especially in medical applications or in the food industry.

A process for the chemical modification of PES membranes includes that the membrane is exposed to strong oxidants (e.g., sodium hypochlorite) in the heat becomes. A disadvantage of this process is the use of environmentally hazardous oxidizing agents. In this case, at least partial depolymerization can be expected.

A disadvantage of (1) sulfonations by treatment of PES and PSf membranes with chlorosulfonic acid in tetrachloromethane or with oleum in methylene chloride, (2) chloromethylations with chloromethyl methyl ether in the presence of a tin catalyst, and (3) carboxylations by butyllithium and CO₂ is the use of expensive, toxic and/or carcinogenic reagents and solvents. In addition, after lithiation with butyllithium, various subsequent reactions are carried out, for example with amines or epoxides.

In an aspect, the present disclosure provides methods of manufacture and optimization of membranes for use in a protein separation system (i.e., a wet grain mill process). These membranes may comprise charged membranes. Charged membranes may comprises membranes that have been modified or created to selectively reject or permeate target solutes depending on characteristics comprising solute size, solute overall charge, and solute polarity.

The mechanism of filtration through the ultrafiltration (UF) and microfiltration (MF) membranes may typically based on size exclusion, where the physical size of the pore determines the rejection of the membrane to the substances of a given molecular weight. Addition of the charged groups to the surface of the membrane or inside of the pore may render the membrane more hydrophilic (potentially reducing fouling) and allow for Donnan exclusion of like-charged species. Charged membranes may exhibit improved retention beyond the membrane molecular cut off, while allowing passage of unwanted small uncharged and charged impurities. In some embodiments, charged based protein and solutes fractionation may be enabled through the use of charged membranes, at higher fluxes as compared to membranes typically selected for those applications.

In one aspect, the present disclosure provides membranes designed for increased rejection or selective fractionation of proteins and components resulting from processing wet mill grain feeds. One or more characteristics of the membranes may comprise the improvement of the rejection or fractionation ability of a membrane beyond the size exclusion of a pore of a given size and addition of electrophoretic exclusion (Donnan exclusion) to the lumen of the pore or surface of the membrane. For the Donnan exclusion to be realized, one needs to operate the feed solution of the proteins at the pH below the isoelectric point (protein to be protonated) and the membrane to be charged positively and the opposite for the pH above the isoelectric point where proteins shall be deprotonated and show net negative charge. For the adsorption of the protein on the surface of the membrane to be achieved, the opposite charges need to be generated in the membrane and protein solutions.

Non-limiting examples of the methods to introduce the charge to the membrane may include; (1) introduction of charge to the casting polymer (sulfonated polysulfone membranes, sulfonated polyether sulfone, copolymers of membrane forming polymer with charged polymers, membranes formed from charged polymers, membrane formed with charged polymeric additives etc.), (2) covalently attached and/or absorbed charged polymer coatings, (3) covalently attached and absorbed polymer coatings with chemical groups characterized by negative charge, (4) covalently attached and absorbed polymer coatings with chemical groups characterized by negative charge, (5) covalently attached and absorbed polymer coatings with zwitterionic properties, (6) chemical treatments of the surface of the membrane, and (7) covalently attach the functionality of choice by impregnation of membrane with low or high molecular weight compound baring the functionality of choice and irradiation of the membrane surface using ionizing radiation (triggering covalent attachment of the functional charged group of choice).

Net negative charge may be introduced to the surface of the membrane and the lumen of the pore by utilization of a sulfonated base membrane forming polymer (PES, PS, PVDF, CA, PAN, etc). In some cases, sulfonated polyether sulfone may be obtained by reaction of polyether sulfone with sulfuric acid. The negatively charged ultrafiltration membranes may be produced by mixing sulfonated polymers mixed with non-sulfonated poly(aryl ethers) to form a polymer blend, which may be then dissolved in solvents and cast via the phase inversion process. In some cases, the membranes may be characterized by molecular weight cut offs (MWCO) ranging from about 3,000 to about 300,000 Daltons, making them ideal for protein separation. The design of the membrane with the negative zeta potential allows for more efficient separation and fractionation of the proteins characterized by a similar charge. This combination allows for minimal fouling to occur due to repulsion forces between the surface of the membrane and the protein. Membranes characterized by negative surface zeta-potential can be prepared using polymers, copolymers, and blends of polymers. The functional group may be selected from any permanently charged chemical groups or polarizable chemical groups, which enables pH tunability of the membrane charge. In some embodiments, the net negatively charged (zeta potential) membrane may be produced using chlorinated polyvinylchloride, as a membrane forming polymer providing the net negative charge on the surface. It can be used as a stand-alone membrane forming material and in presence of additives providing negative and/or positive charges to the membrane surface and pores. Net positive charge can also be introduced to the surface of the membrane and the lumen of the pore by utilization of proper polymers, copolymers, or combinations thereof.

In some cases, generation of charged membranes characterized by either positive or negative charge may depend on functionality of choice, which may be delivered to the surface of the membrane. In some cases, the functionality may be delivered to the membrane by covalent bonding, impregnation of membrane with low or high molecular weight compound baring the functionality of choice, or irradiation of the membrane surface using ionizing radiation. In some cases, a process for the modification of polymer membranes comprises a direct modification of the membrane polymer with low molecular weight compounds using high-energy electron radiation.

In some cases, a microporous membrane having a polymer crosslinked to the surface may be produced by following steps: a) providing a microporous starting membrane, b) impregnating the membrane with an impregnating solution comprising a solvent and a dissolved or dispersed polymer (bearing the charged chemical groups of choice within its repeating units or the end groups) therein to provide an impregnated membrane, and c) irradiating the impregnated membrane with electron beam radiation to provide a microporous membrane having on its surface the electron beam crosslinked polymer fixed thereto.

In some cases, a functional charged membrane may be produced by (1) an ion beam ionization step of irradiating a polymer film on the microporous substrate with high-energy heavy ions at a rate of about 10⁴/cm² to about 10¹⁴/cm² to form active species in the film to bind to the substrate covalently; and (2) a graft polymerization step subsequent to the ion irradiation step wherein one or more monomers selected from the group consisting of useful functional group-containing monomers are added so that the monomers are graft-polymerized with the film substrate.

In some cases, the process producing a functional charged membrane may comprise radiation triggered chemical reactions in which a membrane may be impregnated with monomers and irradiated with high-energy radiation, grafting the monomers onto the membrane substrate forming a film of an ionic compound. In some cases, polymeric microporous support may be treated with an ionizing radiation of energy of about 15 to about 50,000 electron volt (eV) at a dosage of at least about 0.01 watt second/cm² before and/or during application of the coating material bearing charged functionality.

In some cases, microporous polymers used as substrates comprise polyethersulfone (PES), polysulfones (PSF), polyvinylidene fluoride (PVDF) or polyacrylonitrile (PAN).

Non-limiting examples of the functionalities may include —COOR, —CONR₂, —OR, —SO₃R, —P(O)(OR)₂, —PO₃R, —P(O)(OR), —P(OR)₂, —SH, —OH, —PR₃⁺, —NR₂, and —NR₃⁺, wherein R comprises Alkyl-, Aryl-, or Aralkyl groups.

Low molecular weight compounds, as used herein, refer to substances that are not present as a polymer or oligomer. In some embodiments, the low molecular weight compounds have a molecular weight less than about 5000 g/mol. In some embodiments, the low molecular weight compounds may have at least one of the functional groups selected from the group consisting of —COOR, —CONR₂, —OR, —SO₃R, —P(O)(OR)₂, PO₃H, P(O)(OR), P(OR)₂, —OH, —SH, PR₃⁺, —NR₂, —NR₃⁺, wherein R may be the same or different and may be independently hydrogen atoms, alkyl, aryl, or aralkyl groups. In some embodiments, the functional groups may be present in any combination and frequency in the compound. In some embodiments, the compound may be saturated aliphatic or aromatic organic compounds. In some embodiments, the low molecular weight compounds have about 2 to about 30 carbon atoms. In some embodiments, the low molecular weight compounds have about 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms. In some embodiments, the low molecular weight compounds have about 2 to 30, 3 to 29, 4 to 28, 5 to 27, 6 to 26, 7 to 25, 8 to 24, 9 to 23, 10 to 22, 11 to 21, 12 to 20, 13 to 19, 14 to 18, 15 to 17, or 16 to 30 carbon atoms. In some embodiments, the low molecular weight compounds may be aliphatic compounds having about 2 to about 10 carbon atoms. In some embodiments, the low molecular weight compounds may be aliphatic compounds having about 2 to about 8 carbon atoms. In some embodiments, the low molecular weight compounds may be aliphatic compounds having about 2, 3, 4, 5, 6, 7, 8, 9, or 10 carbon atoms. In some embodiments, the low molecular weight compounds may be aliphatic compounds having about 2 to 10, 3 to 9, 4 to 8, 5 to 7, 6 to 10 carbon atoms. In some embodiments, the low molecular weight compounds may be the aromatic compounds having about 6 to about 20 carbon atoms. In some embodiments, the low molecular weight compounds may be the aromatic compounds having about 6 to about 16 carbon atoms. In some embodiments, the low molecular weight compounds may be the aromatic compounds having about 6, 7, 8, 9, 10, 11, 12, 13, 14, 15, 16, 17, 18, 19, or 20 carbon atoms. In some embodiments, the low molecular weight compounds may be the aromatic compounds having about 6 to 20, 7 to 19, 8 to 18, 9 to 17, 10 to 16, 11 to 15, 12 to 14, or 13 to 20 carbon atoms.

The term “aliphatic compounds” as used herein refers to the compounds which have no carbon-carbon double bonds or triple bonds or aromatic bonds but in which only carbon-carbon single bonds are present (in combination with the functional groups disclosed herein). The term “aromatic organic compounds” as used herein refers to the organic compounds having an aromatic moiety, such as a phenyl group. However, it may not be excluded that, in addition to such an aromatic fraction, further, for example aliphatic side chains are present. In order to be suitable for functionalizing and modifying the polymer membrane, the low molecular weight compounds have functional groups, so that the desired modification may be made possible by the irradiation treatment to be used according to the disclosure herein. Suitable functional groups according to the disclosure herein comprise —COOR, —CONR₂, —OR, —SO₃R, —P(O)(OR)₂, —PO₃R, —P(O)(OR), —P(OR)₂, —SH, —OH, —PR₃⁺, —NR₂, or —NR₃⁺, wherein R may be the same or different and may be independently hydrogen, alkyl, aryl, or aralkyl groups.

Alkyl groups as used herein define their saturated moieties, which have only carbon atoms and hydrogen atoms. In some embodiments, the carbon number may be 1 to 15. In some embodiments, the carbon number may be 1 to 10. In some embodiments, the carbon number may be 1 to 4. In some embodiments, the carbon number may be 1, 2, 3, 4, 5, 6, 7, 8, 9, 10, 11, 12, 13, 14, or 15. In some embodiments, the carbon number may be 1 to 15, 2 to 14, 3 to 13, 4 to 12, 5 to 11, 6 to 10, 7 to 9, or 8 to 15. In some embodiments, the alkyl groups may be straight-chained, branched, or cyclic. In some embodiments, the alkyl groups may be straight-chain or branched groups. In some embodiments, the aryl groups may be aromatic groups having 6 or more carbon atoms. In some embodiments, the aryl groups may be aromatic groups having 6 to 20 carbon atoms. In some embodiments, the aryl groups may be aromatic groups having 6, 7, 8, 9, 10, 11, 12, 13, 14, 15, 16, 17, 18, 19, or 20 carbon atoms. In some embodiments, the aryl groups may be aromatic groups having 6 to 20, 7 to 19, 8 to 18, 9 to 17, 10 to 16, 11 to 15, 12 to 14, or 13 to 20 carbon atoms. In some embodiments, the aryl groups may have only carbon atoms and hydrogen atoms. In some embodiments, the aryl group may be a phenyl group. In some embodiments, the aralkyl groups may be the combination of the alkyl groups disclosed herein and the aryl groups disclosed herein. In some embodiments, R may be selected from hydrogen atoms and alkyl groups as disclosed herein: —COOR, —CONR₂, —OR, —NR₂, —B(O)(OR)₂, —CONR₂, wherein R is as disclosed herein. In some embodiments, R may be selected from a hydrogen atom and alkyl groups having 1 to 4 carbon atoms. In some embodiments, the alkyl groups may be methyl or ethyl group.

In some embodiments, the low molecular weight compounds may have at least one of the following functional groups: —COOR, —CONR₂, —OR, —NR₂, —B(O)(OR)₂, —CONR₂, wherein R is as disclosed herein. In some embodiments, R may be selected from a hydrogen atom and alkyl groups having 1 to 4 carbon atoms. In some embodiments, the alkyl groups may be methyl or ethyl group.

In some embodiments, the low molecular weight compounds suitable for modifying polymer membranes comprise benzoic acid, malonic acid, phenylphosphonic acid, taurine, toluenesulfonic acid, glycerol, ethylamine, triethylamine, methylmalonic acid, naphthalenedisulfonic acid, phosphorylcholine, diethylphosphoramidate, glutamine, glucose, phosphonopropionic acid, or mixtures thereof. In some embodiments, the compounds may carry several of the different functional groups combined.

In some cases, the membranes may be wetted with aqueous solutions of the low molecular weight compounds. In some embodiments, the low molecular weight compounds according to the present disclosure are present in proportions of about 0.1 to about 5% by weight, dissolved in deionized water. In some embodiments, the low molecular weight compounds according to the present disclosure are present in proportions of about 0.1 to about 0.75% by weight, dissolved in deionized water. In some embodiments, the low molecular weight compounds according to the present disclosure are present in proportions of about 0.1, 0.5, 1. 1.5, 2, 2.5, 3, 3.5, 4, 4.5, or 5% by weight, dissolved in deionized water. In some embodiments, the low molecular weight compounds according to the present disclosure are present in proportions of about 0.1 to 5, 0.5 to 4.5, 1 to 4, 1.5 to 3.5, 2 to 3, or 2.5 to 5% by weight, dissolved in deionized water. The solution may also contain a mixture of the low molecular weight compounds in order to be tailored specifically to the subsequent application to achieve properties. In some embodiment, the aqueous solution for wetting treatment consists of water and one or more of the low molecular weight compounds used for the modification the polymer membrane. In some embodiments, the water is deionized water. Wetting may be carried out by brief immersion of the polymer membrane in the aqueous or organic solution, followed by removal of the polymer membrane and optionally rinsing of the polymer membrane. Then a drying can take place before the electron beam treatment. In some embodiments, the drying may be performed at temperatures of up to about 120° C. In some embodiments, the drying may be performed in air or in natural gas atmosphere. This may be followed by the radiation treatment. In some embodiments, the radiation treatment may be carried out in an atmosphere with reduced oxygen content. In some embodiments, the wetted membrane may be exposed in a nitrogen atmosphere with oxygen contents less than about 500 parts per million (ppm) high-energy radiation. In some embodiments, the wetted membrane may be exposed in a nitrogen atmosphere with oxygen contents less than about 100 ppm high-energy radiation. In some embodiments, the wetted membrane may be exposed in a nitrogen atmosphere with oxygen contents less than about 10 ppm high-energy radiation. In some embodiments, the wetted membrane may be exposed in a nitrogen atmosphere with oxygen contents less than about 10, 50, 100, 150, 200, 250, 300, 350, 400, 450, or 500 ppm high-energy radiation. In some embodiments, the wetted membrane may be exposed in a nitrogen atmosphere with oxygen contents less than about 10 to 500, 50 to 450, 100 to 400, 150 to 350, 200 to 300, or 250 to 500 ppm high-energy radiation. The radiation comprises an electron radiation. In some embodiments, the energy dose required for the modification according to the present disclosure may be in the range of about 10 to about 200 kilogray (kGy). In some embodiments, the energy dose may be in the range of about 50 to about 200 kGy. In some embodiments, the energy dose required for the modification according to the present disclosure may be in the range of about 10, 25, 50, 75, 100, 125, 150, 175, or 200 kGy. In some embodiments, the energy dose may be in the range of about 10 to 200, 25 to 175, 50 to 150, 75 to 125, or 100 to 200 kGy.

In some cases, the modified polymer membranes may be characterized by an improved surface hydrophilicity as an additional result to the introduction of charged groups. In some cases, the polymer membranes may be selected from the group consisting of PES, PAN, PSF, PVDF, or mixtures thereof. In some cases, the base material for the polymer membrane may not be critical to the coating processes.

In some embodiments, the process for the modification of polymer membranes may be impregnated with an aqueous solution of low molecular weight compounds, irradiated with ionizing radiation and thus covalently functionalized. In some embodiments, the low molecular weight compounds may be saturated aliphatic or aromatic organic compounds. In some embodiments, the low molecular weight compounds have a molecular weight less than about 5000 g/mol. In some embodiments, the low molecular weight compounds may have at least one of the functional groups selected from the group consisting of —COOR, —CONR₂, —OR, —SO₃R, —P(O)(OR)₂, PO₃H, P(O)(OR), P(OR)₂, —OH, —SH, PR₃⁺, —NR₂, —NR₃⁺, wherein R may be the same or different and may be independently hydrogen atoms, alkyl, aryl, or aralkyl groups. In some embodiments, the functional groups may be present in any combination and frequency in the compound, wherein the ionizing radiation may be electron radiation irradiated with about 50 to about 250 keV and in a dose range between about 10 to about 200 kGy. In some embodiments, aqueous solutions of the low molecular weight compounds may be used in which the molecules are present in proportions by weight of about 0.1 to about 5 wt %. In some embodiments, process for the modification of polymer membranes may use polyethersulfone, polysulfone, polyvinylidene fluoride or polyacrylonitrile for the modification. In some embodiments, the process may use an electron radiation with an energy of about 140 to about 180 keV and in a dose range of about 50 to about 200 kGy. In some embodiments, the irradiation may take place in an inert atmosphere having an oxygen content less than about 500 ppm. In some embodiments, the irradiation may take place in an inert atmosphere having an oxygen content less than about 100 ppm. In some embodiments, the irradiation may take place in an inert atmosphere having an oxygen content less than about 10 ppm. In some embodiments, the irradiation may take place in an inert atmosphere having an oxygen content less than about 10, 50, 100, 150, 200, 250, 300, 350, 400, 450, or 500 ppm. In some embodiments, the irradiation may take place in an inert atmosphere having an oxygen content less than about 10 to 500, 50 to 450, 100 to 400, 150 to 350, 200 to 300, or 250 to 500 ppm. In some embodiments, the low molecular weight compound may be selected from benzoic acid, malonic acid, phenylphosphonic acid, taurine, toluenesulfonic acid, glycerol, ethylamine, triethylamine, methylmalonic acid, naphthalenedisulfonic acid, phosphorylcholine, diethylphosphoramidate, glutamine, glucose, phosphonopropionic acid or mixtures thereof. In some embodiments, the polymer membrane may be ultrafiltration membrane or microfiltration membrane.

In an aspect, the present disclosure provides modified or designed membranes for processing wet mill grain feeds. One or more characteristics of the membranes may comprise pore size, pore charge, membrane surface modification, membrane composite material, membrane base layer, membrane structural design. In addition, certain membrane characteristics may be specifically chosen with respect to one or more other membranes within the system. For example, the pore size of a first membrane may be selected from a range based on pore size of a second membrane, given a flow or flux value of the designed system.

In some embodiments, the membrane has a negative charge from about 0.05 milliequivalents per square meter to about 2.5 milliequivalents per square meter. In some embodiments, the membrane has a negative charge of at least about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 milliequivalents per square meter. In some embodiments, the membrane has a negative charge of at most about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 milliequivalents per square meter. In some embodiments, the membrane has a negative charge of from about 0.05 to 2.5, 0.1 to 2.4, 0.2 to 2.3, 0.3 to 2.2, 0.4 to 2.1, 0.5 to 2.0, 0.6 to 1.9, 0.7 to 1.8, 0.8 to 1.7, 0.9 to 1.6, 1.0 to 1.5, 1.1 to 1.4, or 1.2 to 1.3 milliequivalents per square meter. In some embodiments, the membrane has protein sieving coefficient of about 0.001 to about 0.005. In some embodiments, the membrane has protein sieving coefficient of at least about 0.001, 0.002, 0.003, 0.004, or 0.005. In some embodiments, the membrane has protein sieving coefficient of at most about 0.001, 0.002, 0.003, 0.004, or 0.005. In some embodiments, the membrane has protein sieving coefficient of from about 0.001 to 0.005, 0.002 to 0.004, or 0.003 to 0.005. In some embodiments, the membrane has negative zeta potential up to about −60 mV.

In some embodiments, the membrane has a positive charge from about 0.05 milliequivalents per square meter to about 2.5 milliequivalents per square meter. In some embodiments, the membrane has a positive charge of at least about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 milliequivalents per square meter. In some embodiments, the membrane has a positive charge of at most about 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5 milliequivalents per square meter. In some embodiments, the membrane has a positive charge of from about 0.05 to 2.5, 0.1 to 2.4, 0.2 to 2.3, 0.3 to 2.2, 0.4 to 2.1, 0.5 to 2.0, 0.6 to 1.9, 0.7 to 1.8, 0.8 to 1.7, 0.9 to 1.6, 1.0 to 1.5, 1.1 to 1.4, or 1.2 to 1.3 milliequivalents per square meter. In some embodiments, the membrane has protein sieving coefficient of about 0.001 to about 0.005. In some embodiments, the membrane has protein sieving coefficient of at least about 0.001, 0.002, 0.003, 0.004, or 0.005. In some embodiments, the membrane has protein sieving coefficient of at most about 0.001, 0.002, 0.003, 0.004, or 0.005. In some embodiments, the membrane has protein sieving coefficient of from about 0.001 to 0.005, 0.002 to 0.004, or 0.003 to 0.005. In some embodiments, the membrane has positive zeta potential up to about 60 mV.

In some embodiments, the charged membranes may be used in a series of membrane fractionation steps. In some embodiments, the charged membranes may be used in one or more membrane fractionation steps.

Additional membrane characteristics and combinations of characteristics among one or more membranes of a system can be shown in the listed examples of the present disclosure.

### Operating Conditions and Parameters

In an aspect, the present disclosure provides operating conditions and parameters that are particularly useful for protein separation from wet mill grain processes (e.g., corn, pea and soy feed streams).

In an aspect, the present disclosure provides methods and systems which facilitate recovery of valuable ingredients comprising proteins from a grain process (e.g., a wet mill grain process). The methods and systems may comprise the use of a membrane fractionation process. Membrane fractionation may comprise a series of membrane fractionation steps (e.g., greater than or equal to about 2, 3, 4, 5, 6, 7, 8, 9, 10 membrane fractionation steps, or more) in which the membrane materials are selected to maximize the recovery of target protein species and ensure high efficiency and selectivity in rejection. Proper selection of membrane fractionation steps can provide selective protein fractionation.

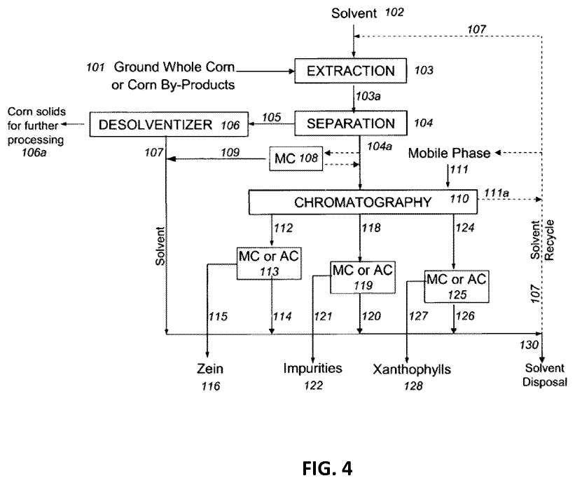

In an aspect, the present disclosure provides a process for recovery of a species (e.g., gluten, prolamin, or other protein) that may not, or need not, require the addition of other chemicals (e.g., alcohol). The process may not, or need not, involve the use of an organic solvent, such as ethanol or other aliphatic alcohols, to recover gluten and/or prolamin proteins (FIG. 4). In another aspect, a process of the present disclosure may not, or need not, require a pH adjustment step to recover gluten and/or prolamins proteins. In some cases, a process of the present disclosure may require a pH adjustment step to recover target proteins.

The protein recovery process and the equipment of the present disclosure are relatively simple and readily scalable, as compared to other process technologies that have been evaluated or commercialized to recover low molecular weight glutens and prolamins from wet mill grain process streams that contains fine particulates, colloidal or soluble forms of proteins. Thus, the simplicity and scalability of the protein recovery process provides an economic route to recover valuable protein streams, in particular lower molecular weight glutens and prolamins, as well as providing an economic route to produce high purity prolamin products. The protein recovery process offers the opportunity to develop new, higher volume markets for these prolamin products, e.g., the production of biodegradable plastic products.

In some aspects, the protein recovery process of this disclosure may be collocated and/or integrated into one or more commercial wet mill grain processes. In some embodiments, the wet mill grain processes may produce streams comprising fine particulate, colloidal or soluble proteins. In some embodiments these streams from wet mill grain processes may be feed streams for the separation processes of the present disclosure.

In an aspect, the present disclosure provides a method of producing high-value gluten and prolamin products, and pure prolamin products, from streams comprising proteins present as fine particulates, colloidal species or soluble species. In some cases, these proteins present as fine particulates, colloidal species or soluble species are of low value or are lost to waste treatment without the process of the present disclosure.

In an aspect, the present disclosure provides a method of recovering fine particulate or protein species from a feed from a wet mil grain process. In some cases, the method comprises (a) subjecting the feed from the wet mill process to a first separation process utilizing a first membrane under conditions effective to provide a first retentate stream comprising one or more higher molecular weight proteins having an average molecular weight of greater than 75 kDa and a first permeate stream comprising one or more lower molecular weight proteins having an average molecular weight of less than 75 kDa, and (b) subjecting the first permeate stream to a second separation process utilizing a second membrane under conditions effective to provide a second retentate stream comprising glutens and a second permeate stream comprising prolamins.

When a feed inlet stream (e.g. the feed stream) enters a membrane module (e.g. an ultrafiltration or nanofiltration process) it may be separated into two streams: a permeate stream which passes through the membrane, and a retentate stream which does not pass through the membrane and is retained on the feed side of the membrane.

In some cases, the first membrane may comprise a microporous membrane, an ultrafiltration membrane, or a nanofiltration membrane. In some cases, the second membrane may comprise an ultrafiltration membrane or a nanofiltration membrane. In some cases, one or more of the first membrane, second membrane, microporous membrane, ultrafiltration membrane, and nanofiltration membrane may comprise at least one charged membrane. One or more of the first membrane, second membrane, microporous membrane, ultrafiltration membrane, and nanofiltration membrane may comprise at least one spiral wound membrane element. In some cases, the first and/or second separation process may utilize at least one spiral wound membrane element. In some cases, one or both of the first and second separation processes may utilize at least two membrane elements. The at least two membrane elements may be in parallel. Alternatively, the at least two membrane elements may be in series.

In some cases, the first separation process may comprise a first membrane. In some cases, the first separation product and/or the first membrane may produce a first retentate stream. In some cases, the first retentate stream may comprise a first product stream. In some cases, the first separation process and/or the first membrane may produce a permeate stream (e.g., a first permeate stream).

In some cases, the permeate stream (e.g., first permeate stream) is subjected to a second separation process comprising a second membrane. The second separation process may produce a second retentate stream. The second retentate stream may comprise a second product stream. The second separation process may produce a second permeate stream. The second permeate stream may comprise a third product stream.

In some cases, the feed stream may comprise a first set of proteins. The first set of proteins may comprise one or more higher molecular weight proteins. These higher molecular weight proteins may comprise glutens. In some cases, the first set of proteins may have an average molecular weight of about 2 kilodalton (kDa) to about 3,000 kDa. In some cases, the first set of proteins may have an average molecular weight of about 2 kDa to about 10 kDa, about 2 kDa to about 50 kDa, about 2 kDa to about 75 kDa, about 2 kDa to about 100 kDa, about 2 kDa to about 150 kDa, about 2 kDa to about 200 kDa, about 2 kDa to about 500 kDa, about 2 kDa to about 1,000 kDa, about 2 kDa to about 3,000 kDa, about 10 kDa to about 50 kDa, about 10 kDa to about 75 kDa, about 10 kDa to about 100 kDa, about 10 kDa to about 150 kDa, about 10 kDa to about 200 kDa, about 10 kDa to about 500 kDa, about 10 kDa to about 1,000 kDa, about 10 kDa to about 3,000 kDa, about 50 kDa to about 75 kDa, about 50 kDa to about 100 kDa, about 50 kDa to about 150 kDa, about 50 kDa to about 200 kDa, about 50 kDa to about 500 kDa, about 50 kDa to about 1,000 kDa, about 50 kDa to about 3,000 kDa, about 75 kDa to about 100 kDa, about 75 kDa to about 150 kDa, about 75 kDa to about 200 kDa, about 75 kDa to about 500 kDa, about 75 kDa to about 1,000 kDa, about 75 kDa to about 3,000 kDa, about 100 kDa to about 150 kDa, about 100 kDa to about 200 kDa, about 100 kDa to about 500 kDa, about 100 kDa to about 1,000 kDa, about 100 kDa to about 3,000 kDa, about 150 kDa to about 200 kDa, about 150 kDa to about 500 kDa, about 150 kDa to about 1,000 kDa, about 150 kDa to about 3,000 kDa, about 200 kDa to about 500 kDa, about 200 kDa to about 1,000 kDa, about 200 kDa to about 3,000 kDa, about 500 kDa to about 1,000 kDa, about 500 kDa to about 3,000 kDa, or about 1,000 kDa to about 3,000 kDa. In some cases, the first set of proteins may have an average molecular weight of about 2 kDa, about 10 kDa, about 50 kDa, about 75 kDa, about 100 kDa, about 150 kDa, about 200 kDa, about 500 kDa, about 1,000 kDa, or about 3,000 kDa. In some cases, the first set of proteins may have an average molecular weight of at least about 2 kDa, about 10 kDa, about 25 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 150 kDa, about 200 kDa, about 500 kDa, or about 1,000 kDa. In some cases, the first set of proteins may have an average molecular weight of at most about 10 kDa, about 25 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 150 kDa, about 200 kDa, about 500 kDa, about 1,000 kDa, or about 3,000 kDa.

In some cases, the first set of proteins may have an average particle size of about 0.001 micrometers (μm) to about 100 μm. The first set of proteins may have an average particle size of about 0.001 μm to about 0.005 μm, about 0.001 μm to about 0.01 μm, about 0.001 μm to about 0.02 μm, about 0.001 μm to about 0.05 μm, about 0.001 μm to about 0.1 μm, about 0.001 μm to about 0.5 μm, about 0.001 μm to about 1 μm, about 0.001 μm to about 5 μm, about 0.001 μm to about 10 μm, about 0.001 μm to about 100 μm, about 0.005 μm to about 0.01 μm, about 0.005 μm to about 0.02 μm, about 0.005 μm to about 0.05 μm, about 0.005 μm to about 0.1 μm, about 0.005 μm to about 0.5 μm, about 0.005 μm to about 1 μm, about 0.005 μm to about 5 μm, about 0.005 μm to about 10 μm, about 0.005 μm to about 100 μm, about 0.01 μm to about 0.02 μm, about 0.01 μm to about 0.05 μm, about 0.01 μm to about 0.1 μm, about 0.01 μm to about 0.5 μm, about 0.01 μm to about 1 μm, about 0.01 μm to about 5 μm, about 0.01 μm to about 10 μm, about 0.01 μm to about 100 μm, about 0.02 μm to about 0.05 μm, about 0.02 μm to about 0.1 μm, about 0.02 μm to about 0.5 μm, about 0.02 μm to about 1 μm, about 0.02 μm to about 5 μm, about 0.02 μm to about 10 μm, about 0.02 μm to about 100 μm, about 0.05 μm to about 0.1 μm, about 0.05 μm to about 0.5 μm, about 0.05 μm to about 1 μm, about 0.05 μm to about 5 μm, about 0.05 μm to about 10 μm, about 0.05 μm to about 100 μm, about 0.1 μm to about 0.5 μm, about 0.1 μm to about 1 μm, about 0.1 μm to about 5 μm, about 0.1 μm to about 10 μm, about 0.1 μm to about 100 μm, about 0.5 μm to about 1 μm, about 0.5 μm to about 5 μm, about 0.5 μm to about 10 μm, about 0.5 μm to about 100 μm, about 1 μm to about 5 μm, about 1 μm to about 10 μm, about 1 μm to about 100 μm, about 5 μm to about 10 μm, about 5 μm to about 100 μm, or about 10 μm to about 100 μm. The first set of proteins may have an average particle size of about 0.001 μm, about 0.005 μm, about 0.01 μm, about 0.02 μm, about 0.05 μm, about 0.1 μm, about 0.5 μm, about 1 μm, about 5 μm, about 10 μm, or about 100 μm. The first set of proteins may have an average particle size of at least about 0.001 μm, about 0.005 μm, about 0.01 μm, about 0.02 μm, about 0.05 μm, about 0.1 μm, about 0.5 μm, about 1 μm, about 5 μm, or about 10 μm. The first set of proteins may have an average particle size of at most about 0.005 μm, about 0.01 μm, about 0.02 μm, about 0.05 μm, about 0.1 μm, about 0.5 μm, about 1 μm, about 5 μm, about 10 μm, or about 100 μm.

In some cases, the first feed stream may comprise a second set of proteins. The second set of proteins may comprise gluten and/or prolamin. The second set of proteins may have an average molecular weight less than the average molecular weight of the first set of proteins. In some cases, the second set of proteins may have an average molecular weight of about 2 kilodalton (kDa) to about 500 kDa. In some cases, the second set of proteins may have an average molecular weight of about 2 kDa to about 5 kDa, about 2 kDa to about 10 kDa, about 2 kDa to about 25 kDa, about 2 kDa to about 50 kDa, about 2 kDa to about 75 kDa, about 2 kDa to about 100 kDa, about 2 kDa to about 150 kDa, about 2 kDa to about 200 kDa, about 2 kDa to about 500 kDa, about 5 kDa to about 10 kDa, about 5 kDa to about 25 kDa, about 5 kDa to about 50 kDa, about 5 kDa to about 75 kDa, about 5 kDa to about 100 kDa, about 5 kDa to about 150 kDa, about 5 kDa to about 200 kDa, about 5 kDa to about 500 kDa, about 10 kDa to about 25 kDa, about 10 kDa to about 50 kDa, about 10 kDa to about 75 kDa, about 10 kDa to about 100 kDa, about 10 kDa to about 150 kDa, about 10 kDa to about 200 kDa, about 10 kDa to about 500 kDa, about 25 kDa to about 50 kDa, about 25 kDa to about 75 kDa, about 25 kDa to about 100 kDa, about 25 kDa to about 150 kDa, about 25 kDa to about 200 kDa, about 25 kDa to about 500 kDa, about 50 kDa to about 75 kDa, about 50 kDa to about 100 kDa, about 50 kDa to about 150 kDa, about 50 kDa to about 200 kDa, about 50 kDa to about 500 kDa, about 75 kDa to about 100 kDa, about 75 kDa to about 150 kDa, about 75 kDa to about 200 kDa, about 75 kDa to about 500 kDa, about 100 kDa to about 150 kDa, about 100 kDa to about 200 kDa, about 100 kDa to about 500 kDa, about 150 kDa to about 200 kDa, about 150 kDa to about 500 kDa, or about 200 kDa to about 500 kDa. In some cases, the second set of proteins may have an average molecular weight of about 2 kDa, about 5 kDa, about 10 kDa, about 25 kDa, about 50 kDa, about 75 kDa, about 100 kDa, about 150 kDa, about 200 kDa, or about 500 kDa. In some cases, the second set of proteins may have an average molecular weight of at least about 2 kDa, about 5 kDa, about 10 kDa, about 25 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 150 kDa, or about 200 kDa. In some cases, the second set of proteins may have an average molecular weight of at most about 5 kDa, about 10 kDa, about 25 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 150 kDa, about 200 kDa, or about 500 kDa.

The present disclosure provides molecular weights as a measurement of size. The molecular weight may be calculated or measured through one or more methods. The molecular weight of a polymer may be determined by various methods such as light scattering, viscometry, SEC etc. In the present disclosure, the molecular weight measurements herein may be determined by column elution spectrophotometry. Alternatively the molecular weight measurements herein may be determined by light scattering.

In some cases, the second set of proteins may have an average particle size of about 1 nanometer (nm) to about 1,000 nm. In some cases, the second set of proteins may have an average particle size of about 1 nm to about 5 nm, about 1 nm to about 10 nm, about 1 nm to about 20 nm, about 1 nm to about 50 nm, about 1 nm to about 100 nm, about 1 nm to about 200 nm, about 1 nm to about 500 nm, about 1 nm to about 1,000 nm, about 5 nm to about 10 nm, about 5 nm to about 20 nm, about 5 nm to about 50 nm, about 5 nm to about 100 nm, about 5 nm to about 200 nm, about 5 nm to about 500 nm, about 5 nm to about 1,000 nm, about 10 nm to about 20 nm, about 10 nm to about 50 nm, about 10 nm to about 100 nm, about 10 nm to about 200 nm, about 10 nm to about 500 nm, about 10 nm to about 1,000 nm, about 20 nm to about 50 nm, about 20 nm to about 100 nm, about 20 nm to about 200 nm, about 20 nm to about 500 nm, about 20 nm to about 1,000 nm, about 50 nm to about 100 nm, about 50 nm to about 200 nm, about 50 nm to about 500 nm, about 50 nm to about 1,000 nm, about 100 nm to about 200 nm, about 100 nm to about 500 nm, about 100 nm to about 1,000 nm, about 200 nm to about 500 nm, about 200 nm to about 1,000 nm, or about 500 nm to about 1,000 nm. In some cases, the second set of proteins may have an average particle size of about 1 nm, about 5 nm, about 10 nm, about 20 nm, about 50 nm, about 100 nm, about 200 nm, about 500 nm, or about 1,000 nm. In some cases, the second set of proteins may have an average particle size of at least about 1 nm, about 5 nm, about 10 nm, about 20 nm, about 50 nm, about 100 nm, about 200 nm, or about 500 nm. In some cases, the second set of proteins may have an average particle size of at most about 5 nm, about 10 nm, about 20 nm, about 50 nm, about 100 nm, about 200 nm, about 500 nm, or about 1,000 nm.

The process of the present disclosure may be applicable to wet mill processes for a broad range of grains, including corn, wheat, barley, rye, sorghum and oats. The prolamins in these grains are zein in corn, gliadin in wheat, hordein in barley, secalin in rye, kafirin in sorghum and avenin in oats. In some aspects, the protein recovery process of the present disclosure may maximize recovery and facilitate selective separation of valuable and previously lost protein species, including prolamins.

In some cases, the second set of proteins (e.g., one or more lower molecular weight proteins) may comprise glutens and/or prolamins. The glutens may be low molecular weight glutens. The glutens may have an average molecular weight of less than or equal to about 200 kDa, 150 kDa, 100 kDa, 90 kDa, 80 kDa, 70 kDa, 60 kDa, 50 kDa, 40 kDa, 30 kDa, or lower. In some embodiments, the glutens may have an average molecular weight of about 200 kDa to about 30 kDa, about 150 kDa to about 40 kDa, about 100 kDa to about 50 kDa, about 90 kDa to about 60 kDa, or about 80 kDa to about 70 kDa.

In some cases, one or more of the first retentate stream, second retentate stream, first permeate stream, and second retentate stream may be one or more recovery stream(s) of low molecular weight proteins. These one or more recovery stream(s) may comprise greater than about 5%, 10%, 15%, 20%, 25%, 30%, 35%, 40%, 45%, 50%, 55%, 60%, 65%, 70%, 75%, 80%, 85%, 90%, 95%, 99%, or greater of the low molecular weight proteins in the feed stream. In some embodiments, the one or more recovery stream(s) may be about 5% to about 99%, about 10% to about 95%, about 15% to about 90%, about 20% to about 85%, about 25% to about 80%, about 30% to about 75%, about 35% to about 70%, about 40% to about 65%, about 45% to about 60%, or about 50% to about 55% of the low molecular weight proteins in the feed stream.

In some cases, the second permeate stream can comprise both precipitated prolamin proteins and prolamin proteins that remain somewhat soluble. In some case, the second set of proteins may comprise soluble prolamin proteins prior to the second separation process (e.g. in the feed or first permeate stream) and precipitated prolamin proteins after the second separation process (e.g., in the second permeate stream). In some cases, the process of the present disclosure further comprises spray drying the entire permeate stream, comprising prolamin proteins. In some cases, spray drying the entire permeate stream may allow for recovery of a more pure prolamin product than without spray drying. In other cases, the process of the present disclosure may further comprise recovering precipitated prolamin proteins using standard separation methods. Such methods may include drying, decanting, filtration, centrifugation, or an additional membrane filtration step. The initial prolamin product may be further purified to prepare a pure prolamin final product, for example by a chromatographic approach or by simple recrystallization of the prolamin.

The feed from the wet mill grain process may be any stream comprising significant levels of proteins. In some cases, the major grain products (germ, starch, gluten meal) may have been removed. The feed may be an overflow stream from a thickener, decanter or centrifuge. The stream may be the filtrate from filter apparatus, e.g. a rotary vacuum filter. As a non-limiting example, the feed stream may contain less than about 5 wt % total solids and less than about 2 wt % suspended solids. In another example, the feed stream may contain less than about 5 wt %, and most preferably less than about 4 wt % total solids.

The process of the present disclosure may handle up to about 100% of the overflow stream. In some cases, only a portion of the overflow stream is sent to the protein recovery process.

The present disclosure provides for at least a first and second membrane. The first and second membranes may comprise one or more membrane elements. An example of a membrane element may include a spiral wound membrane. The advantages of spiral wound membranes may include: significantly higher cross-sectional surface area, continuous processing, flexible and adaptable design based on both morphology and chemistry of substances rejected, wide pH operating range, temperature stability and capability of being formulated with specific surface properties. In some cases, the first and second membranes may comprise one or more charged membranes.

The material selected for the membrane element is very important. The one or more membrane elements may comprise FDA approved components. The membrane elements may have contributed particular advantages to the processes or methods of the present disclosure on the basis of the membrane elements' pore size, chemical properties, processability, and membrane durability to process conditions and fouling rate. The membrane elements may include spacers. The spacers may allow the one or more of the separation processes of the present disclosure to better accommodate solids. In some cases, the membrane elements may comprise about 10 mils to about 270 mils spacer elements. The membrane elements may comprise about 30 mils to about 135 mils spacer elements. The size of the spacer elements may be tailored to the morphology of the solids being separated. The spacer elements of the membranes may improve the ability of the membranes to accommodate pasty and non-amorphous solids. The spacer elements may further retain the surface area advantage of the membrane (e.g., such as a spiral wound membrane), versus a membrane that does not comprise spacer elements (e.g., hollow fiber or tubular membrane).

In an aspect, the protein recovery process may comprise at least two membrane steps. In some cases, a first step of the at least two-step membrane protein recovery process may involve the use of first membrane (e.g., a microporous membrane). The first membrane may be an ultrafiltration membrane. The first membrane may be a spiral wound membrane. The first membrane may be a microporous polymer membrane. The first membrane may have an average pore size between about 1 nanometer (nm) and about 1000 nm. The first membrane may have an average pore size of about 3 nm to about 500 nm. The first membrane may have an average pore size of about 1 nm to about 5 nm, about 3 nm to about 10 nm, about 3 nm to about 20 nm, about 3 nm to about 50 nm, about 3 nm to about 100 nm, about 3 nm to about 250 nm, about 3 nm to about 500 nm, about 5 nm to about 10 nm, about 5 nm to about 20 nm, about 5 nm to about 50 nm, about 5 nm to about 100 nm, about 5 nm to about 250 nm, about 5 nm to about 500 nm, about 10 nm to about 20 nm, about 10 nm to about 50 nm, about 10 nm to about 100 nm, about 10 nm to about 250 nm, about 10 nm to about 500 nm, about 20 nm to about 50 nm, about 20 nm to about 100 nm, about 20 nm to about 250 nm, about 20 nm to about 500 nm, about 50 nm to about 100 nm, about 50 nm to about 250 nm, about 50 nm to about 500 nm, about 100 nm to about 250 nm, about 100 nm to about 500 nm, or about 250 nm to about 500 nm. The first membrane may have an average pore size of about 3 nm, about 5 nm, about 10 nm, about 20 nm, about 50 nm, about 100 nm, about 250 nm, or about 500 nm. The first membrane may have an average pore size of at least about 3 nm, about 5 nm, about 10 nm, about 20 nm, about 50 nm, about 100 nm, or about 250 nm. The first membrane may have an average pore size of at most about 5 nm, about 10 nm, about 20 nm, about 50 nm, about 100 nm, about 250 nm, or about 500 nm. As an example, the first membrane may have an average pore size between about 20 nm to 100 nm.

In some cases, the first membrane may have a molecular weight cut off for the species it fractionates into the first retentate stream and the first permeate stream. The first membrane (which may be a microporous, or ultrafiltration, membrane) may have a pore size that determines a molecular weight cutoff. As a non-limiting example, a molecular weight cutoff of about 100 kDa may correspond to a pore size that permits molecules with a molecular weight of less than about 100 kDa to pass through the membrane. The first membrane may have a molecular weight cutoff greater than or equal to about 5 kilodaton (kDa), 10 kDa, 12 kDa, 15 kDa, 20 kDa, 25 kDa, 30 kDa, 50 kDa, 75 kDa, 100 kDa, 150 kDa, 200 kDa, 250 kDa, 300 kDa, 400 kDa, 500 kDa, 700 kDa, 1,000 kDa, 2,000 kDa, 3,000 kDa, 4,000 kDa, 5,000 kDa, 7,000 kDa, 10,000 kDa, 20,000 kDa, 50,000 kDa, 10,0000 kDa, or greater. The first membrane may have a molecular weight cutoff less than or equal to about 100,000 kDa, 50,000 kDa, 20,000 kDa, 10,000 kDa, 8,000 kDa, 6,000 kDa, 5,000 kDa, 4,000 kDa, 2,000 kDa, 1,000 kDa, 800 kDa, 600 kDa, 400 kDa, 300 kDa, 200 kDa, 150 kDa, 100 kDa, 80 kDa, 50 kDa, 30 kDa, 20 kDa, 15 kDa, 13 kDa, 10 kDa, 6 kDa, or lower. The second membrane may have a molecular weight cut off between any two values described above, such as between about 10 kDa and about 10,000 kDa. In some cases, the first membrane may comprise a microporous structure produced from a polymer, such as polysulfone, polyvinyl difluoride, polyethersulfone, polyacrylonitrile, polyetherimide, or other membrane-forming polymers or polymer blends. The first membrane may include a non-woven polymer backing to enhance the mechanical strength and durability of the microporous membrane.

The first step of the at least two step process, may provide a first retentate stream. In some cases, the first retentate stream may comprise a first set of proteins (e.g., higher molecular weight glutens) and/or particulates. In some cases, the first step may generate a first permeate stream. The first permeate stream may comprise second set of proteins (e.g., lower molecular weight glutens and prolamins). The first retentate stream may comprise a first product stream. The first retentate stream (e.g., first product stream) may be directed to a separation device to facilitate recovery of a first protein product. Alternatively, the first retentate may be returned to the wet mill grain process. In some cases, the wet mill grain process is integrated with the process of the present disclosure. The first retentate stream may be returned to the feed side of one or more units of the wet mill grain process (e.g., a thickener, decanter, centrifuge, or filter). In some cases the first retentate stream may be directed to one or more units to separate out major grain products. In some cases, the first retentate stream is returned to the input of a separation device. The separation device may provide the feed to the membrane recovery process, e.g. the MST Thickener. The permeate stream from the first membrane may be sent to second membrane process of the at least two-step membrane recovery process.

In the first step, the first membrane may reject greater than about 20%, 30%, 40%, 45%, 50%, 55%, 60%, 65%, 70%, 75%, 80%, 85%, 90%, 95%, 99% or more of the insoluble particulates. The insoluble particles may be greater than about 0.5 micrometers in diameter. In some cases, the insoluble particles may have a particle size between about 0.1 micrometer and about 5 micrometers. The first membrane may reject high molecular weight solubles in the range of about 5% to about 70%. The first membrane may reject high molecular weight solubles at a rate greater than about 40%, 45%, 50%, 55%, 60%, 65%, 70%, 75%, 80%, 85%, 90%, 95%, 99% or more, depending on the average pore size of the first membrane. The first and/or second membrane may be designed to provide a pressure-normalized flux. The pressure-normalized flux may ensure operability and aid the process in achieving economic targets. The membranes may be designed to provide long-term stability and durability to cleaning-in-place cycles. The membranes may be designed to remove as much of the insoluble particulates as possible, such as greater than about 40%, 45%, 50%, 55%, 60%, 65%, 70%, 75%, 80%, 85%, 90%, 95%, 99% or more, (e.g., up to about 100%) to reduce the fouling rate of the second membrane (e.g., a nanofiltration membrane) used in the second step.

The second step of the at least two-step membrane protein recovery process may comprise a second membrane. The second membrane may comprise one or more spiral wound nanofiltration membrane. The second membrane may be the same as the first membrane. The second membrane may be an ultrafiltration membrane. The second membrane may have an average pore size less than or equal to the average pore size of the first membrane. The second membrane may have an average pore size of about 1 nanometer (nm) to about 300 nm. The second membrane may have an average pore size of about 1 nm to about 2 nm, about 1 nm to about 3 nm, about 1 nm to about 5 nm, about 1 nm to about 10 nm, about 1 nm to about 20 nm, about 1 nm to about 50 nm, about 1 nm to about 100 nm, about 1 nm to about 300 nm, about 2 nm to about 3 nm, about 2 nm to about 5 nm, about 2 nm to about 10 nm, about 2 nm to about 20 nm, about 2 nm to about 50 nm, about 2 nm to about 100 nm, about 2 nm to about 300 nm, about 3 nm to about 5 nm, about 3 nm to about 10 nm, about 3 nm to about 20 nm, about 3 nm to about 50 nm, about 3 nm to about 100 nm, about 3 nm to about 300 nm, about 5 nm to about 10 nm, about 5 nm to about 20 nm, about 5 nm to about 50 nm, about 5 nm to about 100 nm, about 5 nm to about 300 nm, about 10 nm to about 20 nm, about 10 nm to about 50 nm, about 10 nm to about 100 nm, about 10 nm to about 300 nm, about 20 nm to about 50 nm, about 20 nm to about 100 nm, about 20 nm to about 300 nm, about 50 nm to about 100 nm, about 50 nm to about 300 nm, or about 100 nm to about 300 nm. The second membrane may have an average pore size of about 1 nm, about 2 nm, about 3 nm, about 5 nm, about 10 nm, about 20 nm, about 50 nm, about 100 nm, or about 300 nm. The second membrane may have an average pore size of at least about 1 nm, about 2 nm, about 3 nm, about 5 nm, about 10 nm, about 20 nm, about 50 nm, or about 100 nm. The second membrane may have an average pore size of at most about 2 nm, about 3 nm, about 5 nm, about 10 nm, about 20 nm, about 50 nm, about 100 nm, or about 300 nm. In some cases, the second membrane may have a pore size less than about 50 nm, 45 nm, 40 nm, 35 nm, 30 nm, 25 nm, 20 nm, 18 nm, 16 nm, 14 nm, 12 nm, 10 nm, 9 nm, 8 nm, 7 nm, 6 nm, 5 nm, 4 nm, 3 nm, 2 nm, 1 nm or lower.

In some cases, the second membrane may have a molecular weight cut off for the species it fractionates into the second retentate stream and the second permeate stream. The second membrane may have a molecular weight cutoff greater than or equal to about 2 kilodalton (kDa), 3 kDa, 4 kDa, 5 kDa, 6 kDa, 7 kDa, 8 kDa, 9 kDa, 10 kDa, 12 kDa, 15 kDa, 20 kDa, 25 kDa, 30 kDa, 50 kDa, 75 kDa, 100 kDa, 150 kDa, 200 kDa, 250 kDa, 300 kDa, 400 kDa, 500 kDa, 700 kDa, 1,000 kDa, 2,000 kDa, 3,000 kDa, 4,000 kDa, 5,000 kDa, 7,000 kDa, 10,000 kDa, 20,000 kDa, 50,000 kDa, 10,0000 kDa, or greater. The second membrane may have a molecule weight cutoff less than or equal to about 100,000 kDa, 50,000 kDa, 20,000 kDa, 10,000 kDa, 8,000 kDa, 6,000 kDa, 5,000 kDa, 4,000 kDa, 2,000 kDa, 1,000 kDa, 800 kDa, 600 kDa, 400 kDa, 300 kDa, 200 kDa, 150 kDa, 100 kDa, 80 kDa, 50 kDa, 30 kDa, 20 kDa, 15 kDa, 13 kDa, 10 kDa, 8 kDa, 6 kDa, 5 kDa, 4 kDa, 3 kDa, or lower. The second membrane may have a molecular weight cut off between any two values described above, such as between about 10 kDa and about 10,000 kDa.

Commercially available nanofiltration polymer membranes may include thin film composite membranes. Such thin film composite membranes may comprise a microporous polymer membrane substrate with pore sizes in the range of about 0.05 to about 0.2 microns, and a top nanoporous polymer layer of pore size less than about 20 nm, or molecular weight cut-off (MWCO) less than about 5 kDa. The microporous polymer membrane used as a substrate may be produced from a variety of polymers, such as polysulfone, polyvinyl difluoride, polyethersulfone, polyacrylonitrile, polyetherimide, as well as from other membrane-forming polymers and polymer blends. The top nanoporous polymer layer may be formed by a dip-coating or interfacial polymerization process. The top nanoporous polymer layer may contribute the separation properties of the nanoporous membrane as it selectively rejects ions and soluble compounds based on size and charge. The nanofiltration membrane may also be produced directly using a phase inversion process similar to the process used to produce microporous membranes.

The second separation process (e.g., second membrane) may be designed to provide a second retentate stream comprising a second set of proteins (e.g., lower molecular weight glutens) and a second permeate stream. In some cases, the second permeate stream may comprise comprising greater than about 20%, 25%, 30%, 35%, 40%, 45%, 50%, 55%, 60%, 65%, 70%, 75%, 80%, 85%, 90%, 95%, 99% or more the prolamins from the feed stream. In some cases, the second permeate stream may comprise greater than about 20%, 25%, 30%, 35%, 40%, 45%, 50%, 55%, 60%, 65%, 70%, 75%, 80%, 85%, 90%, 95%, 99% or greater of the second set of proteins (e.g., other lower molecular weight proteins). In some cases, the second permeate stream may comprise at least a portion of non-protein solubles from the feed stream. As discussed above, the removal of the first and/or second set of proteins that comprises glutens may allow a portion of the prolamins in the second permeate stream to precipitate, facilitating their recovery. In some cases, the second retentate stream (e.g. from a nanofiltration membrane) comprising lower molecular weight proteins, may be sent to a separation device to facilitate recovery of a protein product. Alternatively, the second retentate stream (e.g., from a nanofiltration membrane) may be returned to wet mill grain process. The second retentate stream may be directed to the feed side of a thickener, decanter, centrifuge or filter of the wet mill grain process. The second retentate stream may be directed to a further separation device to separate the major grain products from the second retentate stream.

Alternatively, the second retentate stream may be returned to the separation device upstream of the membrane recovery process, e.g. the MST Thickener. The second permeate stream may be sent to a separation device to recover prolamin. The second permeate stream may be spray dried to recover both precipitated prolamins and prolamins that remain somewhat soluble. Precipitated prolamins may be recovered using standard separation methods. Such methods may include centrifuging, decanting, filtering or an additional membrane process. The prolamin (e.g. impure prolamin) may be further purified by methods such as recrystallization or chromatographic separation. Thus, the present disclosure provides a method and process of obtaining a pure prolamin product. The exact method used to purify the prolamin may be specific to the prolamin form. The overflow from the separation device can be returned to the wet mill grain process.

In the second step, the second membrane (e.g., a nanofiltration membrane) may provide a second retentate stream comprising greater than about 20%, 25%, 30%, 35%, 40%, 45%, 50%, 55%, 60%, 65%, 70%, 75%, 80%, 85%, 90%, 95%, 99% or more of the lower molecular weight solubles (e.g., lower molecular weight glutens). The membranes may be designed to provide a pressure-normalized flux to ensure operability and achieve economic targets. The membranes may be designed to provide long-term stability and durability to cleaning-in-place cycles.

The materials of the first membrane (e.g., microporous membrane) and second membrane (e.g., nanoporous membrane) may be the same or different.

The at least two-step membrane process may include an additional (e.g., third) step. The additional step may comprise fractionating the first retentate stream using a third membrane that provides a third retentate stream. This third retentate stream may comprise the highest molecular weight glutens. The third membrane may produce a third permeate stream comprising lower molecular weight glutens.

The process of the present disclosure may be integrated with a wet mill grain preprocess which separates high value agricultural and food products from a broad range of grains, including corn, wheat, barley, rye, sorghum and oats. The process of the present disclosure allows the fractionation and recovery of gluten and prolamin products that are currently lost in the preprocess. In addition, integration with a wet mill grain preprocess may provide additional improvements, including, but not limited to: (i) recovery of the gluten and prolamin products removes organics from the steep and increases upstream capacity; (ii) recovery of the gluten and prolamin products reduces the organic content of the stream sent to waste treatment.

Recovery of the gluten and prolamin products may remove organic compounds from the steep and increase upstream capacity of the wet mill grain process. Recovery of the gluten and prolamin products may remove non-fermantable organic compounds from the steep and increase upstream capacity of the wet mill grain process. Recovery of the gluten and prolamin products may reduce the quantity of organic content of the stream that may be sent to waste treatment.

As indicated above, streams from the at least two-step membrane protein recovery process may be directly returned to the wet mill grain pre-process. Alternatively, the streams may be returned to the wet mill grain pre-process after being subjected to a protein product separation step. Thus, the at least two-step membrane process may be fully integrated into the wet mill grain preprocess and may not, or need not, introduce any new chemicals or by-products into the preprocess. The process may further comprise cleaning one or more membranes in place, allowing additional load to waste treatment. The process may recover enough zein and reduce the costs of treating organic load in wastewater streams to offset the cost of recovery of zein.

Other benefits of the integrating the membrane recovery process into the wet mill grain process may include, but are not limited to: (i) allowing recovery of previously lost protein streams, thus increasing revenue; (ii) providing an option for production of a high purity and high value prolamin products that may expand the commercial applications of these biomaterials; (iii) improving process sustainability and reducing ultimate wastewater impact; (iv) reducing concentration of “non-fermentables” where overflow from the gluten thickener (e.g. MST) is reconstituted into the Steep, ultimately improving plant grind rate.

In some cases, one or more of the product streams may be used to make at least one desirable article. The one or more product streams may be the first or second retentate stream, or the first or second permeate stream.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

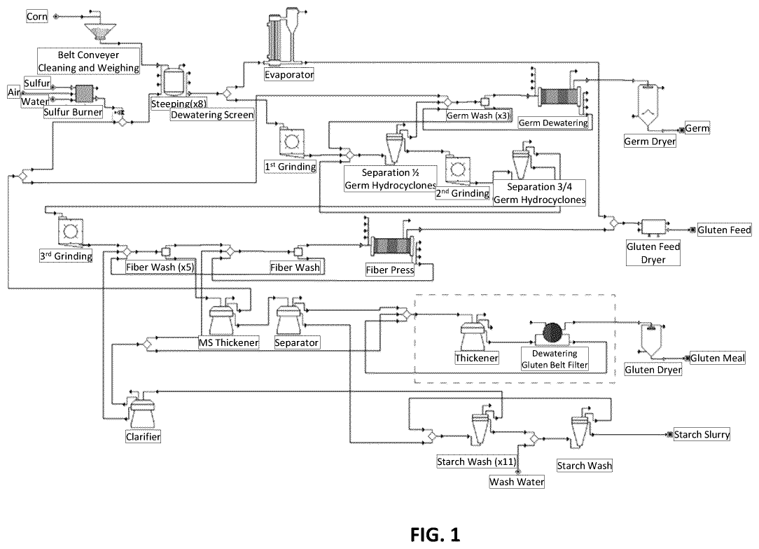

FIG. 1 shows a schematic of a corn wet milling process showing the gluten protein separating step.

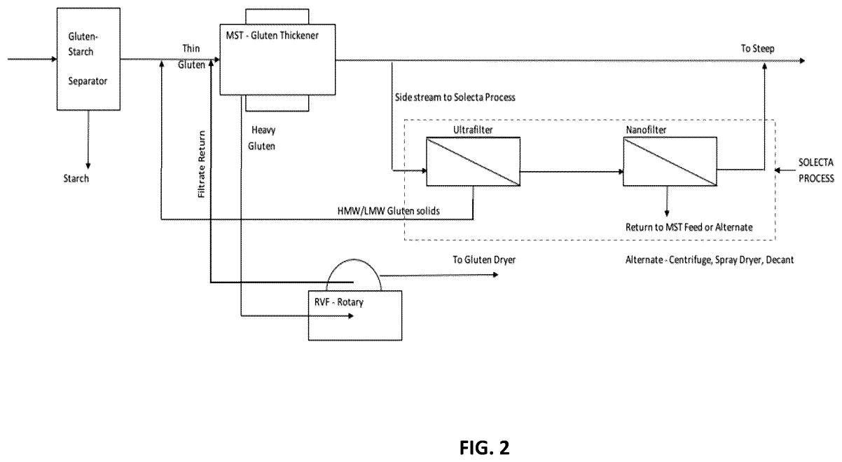

FIG. 2 shows a schematic of a two-step membrane protein recovery process integrated with a wet mill grain process. In FIG. 2, the two-step membrane protein recovery process is integrated downstream of the MST Gluten Thickener. The feed stream 01, which is thin gluten slurry, is sent to a gluten thickener producing an overflow returning to preprocess (Steep), 04 and a heavy gluten stream 05 which is sent to a filter. The filter solids 07 are sent to the gluten dryer and the filtrate 06 returned to the feed side of the gluten thickener. In this disclosure, all or at least a portion of the gluten thickener overflow 04 is sent to the membrane ultrafiltration unit. The permeate from the ultrafiltration unit 08 is sent to a nanofiltration unit. The gluten solids retentate stream 09, comprising insolubles and higher molecular weight glutens, can either be returned to the feed of the gluten thickener or sent to a separation device to recover a protein product. The nanofiltration unit produces a retentate stream comprising lower molecular weight glutens 10 and a permeate stream comprising prolamins 11. The retentate stream 10 can either be returned to the feed side of the gluten thickener or sent to a separation device to recover a protein product. The permeate stream 11, comprising prolamins can be sent to a spray dryer or separation device to recover prolamin products or simply returned to the preprocess (Steep).

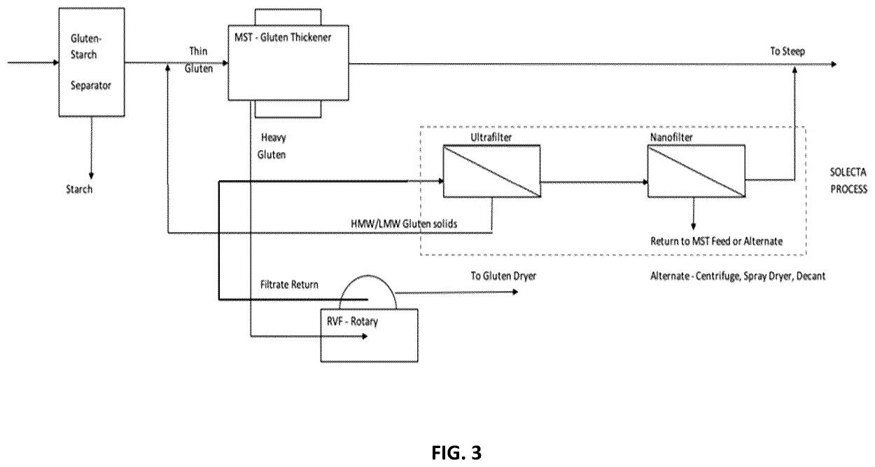

FIG. 3 shows a schematic of a two-step membrane protein recovery process integrated at a mechanical separation device, e.g., a rotary vacuum filter (RVF). The feed stream 21 is fed to a gluten thickener producing an overflow returning to preprocess (e.g. steep) 24 and a heavy gluten stream 25 which is sent to a filter. The filter solids 27 are sent to the gluten dryer and the filtrate 26 sent to the membrane ultrafiltration unit. The permeate from the ultrafiltration unit 28 is sent to a nanofiltration unit. The retentate from the ultrafiltration unit 29, comprising insolubles and higher molecular weight glutens, can either be returned to the feed of the gluten thickener or sent to a separation device to recover a protein product. The nanofiltration unit produces a retentate stream comprising lower molecular weight glutens 30 and a permeate stream comprising prolamins 31. The retentate stream 30 can either be returned to the feed side of the gluten thickener or sent to a separation device to recover a protein product. The permeate stream 31, comprising prolamins can be sent to a spray dryer or separation device to recover prolamin products or simply returned to the preprocess ((e.g., steep).

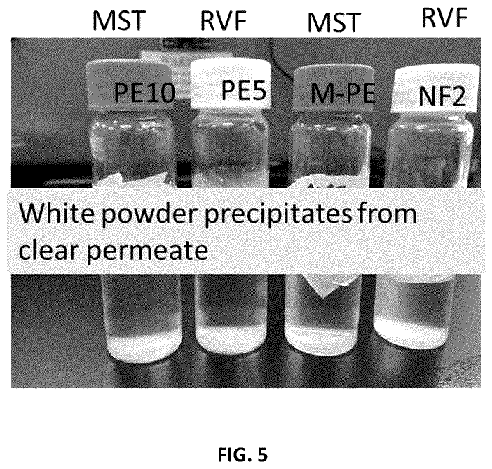

FIG. 5 shows the precipitated prolamin after nanofiltration and dilution.

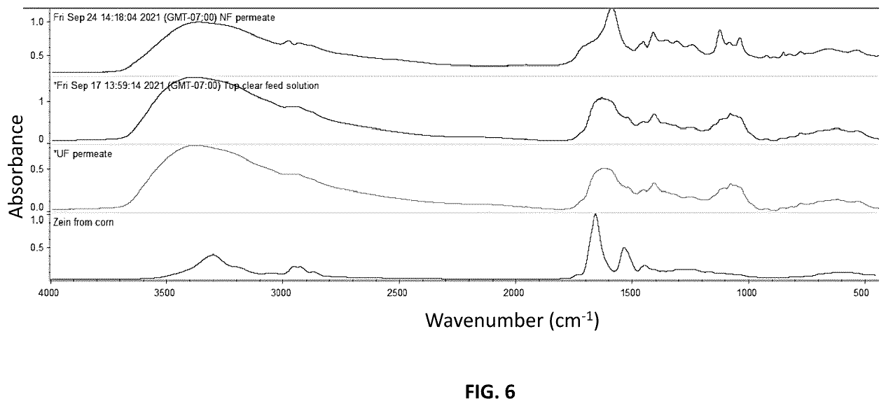

FIG. 6 shows the presence of prolamin within the precipitated white powder of FIG. 5.

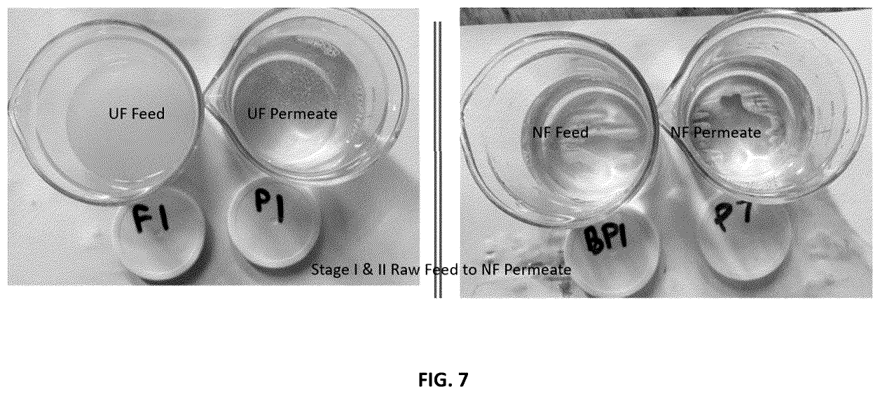

FIG. 7 shows the feed from the thickener, as a gluten source, compared to the permeate from subjecting the feed to a first nanofiltration membrane.

Thus, the present disclosure provides approaches for achieving a membrane fractionation of proteins from a wet mill grain process feed. The membrane fractionation method may produce three streams comprising valuable protein products: (i) a retentate stream from a microporous membrane, comprising higher molecular weight glutens; (ii) a retentate stream from a nanofiltration membrane, comprising lower molecular weight glutens; and (iii) a permeate stream from a nanofiltration membrane, comprising prolamin proteins.

In another aspect, the present disclosure provides a first process of recovering a stream comprising valuable glutens and prolamins, wherein the stream comprising valuable glutens and prolamins is provided by a wet mill grain preprocess that is integrated with the first process.

The disclosure further provides a protein recovery system that is integrated with at least one wet mill grain preprocess.

In some examples, the presence of gluten proteins and other non-protein species stabilized the zein proteins and kept them in solution or perhaps in some colloidal form. By removing the glutens and these other species using the fractionation approach, this stabilization was disrupted, resulting in precipitation of a portion of the zein proteins, thus allowing recovery of the otherwise difficult to recover zein proteins. Prolamins, like zein, generally have poor solubility in water. However, these examples show that the zein may be stabilized in solution by the presence of other organic compounds, both protein and non-protein species, whose removal results in precipitation of the prolamins, thus facilitating their recovery.

In one example, a method of recovering and fractionating valuable protein streams from wet mill grain processes comprises subjecting the overflow or filtrate of a mechanical separation device from a wet grain process to an ultrafiltration process utilizing a microporous membrane under conditions effective to provide an ultrafiltration retentate stream comprising higher molecular weight glutens and a permeate stream comprising colloidal and soluble proteins, comprising lower molecular weight glutens and prolamins; and subjecting the ultrafiltration permeate stream from the microporous membrane process to a nanofiltration process utilizing a nanofiltration membrane under conditions effective to provide a nanofiltration retentate stream comprising lower molecular weight glutens, and a nanofiltration permeate stream comprising prolamins, thus allowing the fractionation (e.g., separation) of higher molecule weight proteins, lower molecular weight proteins and prolamins.

In one example, a method of recovering fine particulate, colloidal and soluble protein species, comprising high and low molecular weight glutens and prolamins, from an aqueous feed from a wet mill grain process comprises: subjecting the feed from the wet grain process to a first separation process utilizing a microporous membrane under conditions effective to provide a retentate stream comprising higher molecular weight proteins and a permeate stream comprising lower molecular weight proteins; and subjecting the permeate stream passing through the first microporous membrane to a second separation process utilizing a second membrane under conditions effective to provide a retentate stream comprising lower molecular weight glutens and a permeate stream comprising prolamins.

In some examples the first separation process comprises utilizing an ultrafiltration membrane. The second separation process may comprise utilizing an ultrafiltration membrane or a nano filtration membrane. In some examples the method comprises a separation process utilizing at least one spiral wound membrane element or at least two spiral wound membrane elements in series or in parallel.

In some examples the lower molecular weight proteins comprise glutens or prolamins.

In another example, a process for recovery of valuable prolamin products from wet mill grain processes comprises subjecting the overflow or filtrate of a mechanical separation device from the wet grain process to an ultrafiltration membrane process utilizing an microporous membrane under conditions effective to provide a ultrafiltration retentate stream comprising higher molecular weight glutens, and a permeate stream comprising fine particulate, colloidal and soluble proteins, including lower molecular weight glutens and prolamins; and subjecting the permeate stream from the microporous membrane process to a nanofiltration process utilizing a nanofiltration membrane under conditions effective to provide a retentate stream comprising lower molecular weight glutens, thus allowing prolamins to be recovered either by direct recovery, such as spray drying, or for the precipitated prolamins by standard separation devices.

In another example, the protein recovery process is integrated with a wet mill grain preprocess and comprises providing a feed from the overflow of a mechanical separation device of the wet grain preprocess to the first process; separating a retentate stream from an microporous membrane process comprising higher molecular weight glutens and returning the retentate stream to the wet mill grain preprocess or recovering a stream containing higher molecular weight glutens using a separation device and returning the overflow or filtrate from the separation device to the wet grain preprocess; sending the permeate stream from the ultrafiltration to nanofiltration; separating a retentate stream from the nanofiltration comprising lower molecular weight glutens and returning the retentate stream to the wet mill grain preprocess or recovering a stream comprising low molecular weight glutens using a separation device and returning the overflow or filtrate from the separation device to the wet grain preprocess; providing a permeate stream comprising prolamins and recovering prolamin products either by direct recovery, such as spray drying, or for precipitated prolamins in the permeate stream recovering the prolamin products using standard separation devices; and returning the liquid stream from the prolamin separation to the wet grain preprocess.

In some examples, the protein recovery process comprises a first process integrated with a wet mill grain preprocess, the first process comprising; providing a wet mill grain preprocess providing a feed from the overflow of a mechanical separation device to the first process; separating a retentate stream comprising higher molecular weight glutens by membrane separation using a microporous membrane; either returning the retentate stream to the wet mill grain preprocess or recovering the higher molecular weight glutens using a separation device and returning a liquid stream from the separation device to the wet mill grain preprocess; sending the permeate stream to a second membrane separation process; separating a retentate stream comprising lower molecular weight glutens using the second membrane; either returning the retentate stream to the wet mill grain preprocess or recovering the high molecular weight glutens using a separation device and returning a liquid stream from the separation device to the wet mill grain preprocess; recovering a permeate stream comprising previously soluble prolamins; recovering the prolamin products using a separation device, such as spray dryer, decanter, centrifuge, filter or membrane element; and returning the liquid stream from the prolamin separation device to the wet mill grain preprocess.

In some examples the wet mill preprocess is a corn, wheat, barley, rye, sorghum or oat wet mill process. In some examples the overflow stream from the mechanical separation device in the wet mill grain preprocess can be the overflow from a thickener, decanter or centrifuge or the filtrate from filtration device, e.g. a rotary vacuum filter.

In some examples the total solids contained in the overhead stream from the wet mill grain preprocess contains less than about 10 wt % solids, less than about 5 wt % solids, less than about 4 wt % solids, less than about 3 wt % solids, less than about 2 wt % solids, less than about 1 wt % solids, less than about 0.5 wt % solids, or less than about 0.1 wt % solids.

In one example the stream from the wet mill grain preprocess is in fluid communication with a microporous membrane in the first process and the retentate stream from the ultrafiltration step of the first process is returned to the wet mill grain preprocess. The permeate stream from ultrafiltration step in the first process is in fluid communication with a nanofiltration membrane in the first process. The retentate stream from the nanofiltration membrane in the first process is in fluid communication with the wet mill grain preprocess. A protein product comprising higher molecular weight proteins is recovered from the ultrafiltration retentate and a liquid stream returned to the wet mill grain process. A protein product comprising lower molecular weight proteins is recovered from the nanofiltration retentate and a liquid stream returned to the wet mill grain process. Proteins comprising prolamins in the permeate stream from the nanofiltration membrane are recovered and the liquid stream is returned to the wet mill grain preprocess. Recovery of a stream comprising prolamins is increased by removal of a stream comprising other low molecular weight proteins, comprising glutens. The recovered protein species comprise glutelins, prolamins and glutens. Recovered non-protein species comprise carbohydrates, starches, enzymes, alcohols, aldehydes, fats or other low molecular weight organic species. The streams comprising solid protein species are recovered by centrifugation, spray-drying, decanting, filtration or an additional membrane step. The recovered prolamin can be further purified to provide high purity prolamin products. In some cases the recovered prolamin may be further purified to provide a high purity prolamin product with an average molecular weight of greater than about about 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 90 kDa, 100 kDa, 120 kDa, 150 kDa, or greater.

In some examples the microporous membrane is a low fouling spiral wound membrane comprising at least one material comprising polysulfone, polyvinyl difluoride, polyethersulfone, polyacrylonitrile, or polyetherimide. The nominal pore size range for the microporous membrane is equal to or between about 0.01 microns and about 0.75 microns. In other examples, the nominal pore size may be equal to or between about 0.02 microns and about 0.50 microns, equal to or between about 0.05 microns to about 0.20 microns, or equal to or between about 0.05 microns to about 0.10 microns. The feed pressure to the ultrafiltration membranes of this example is equal to or between about 15 psig and about 60 psig. In other examples, the feed pressure may be equal to or between about 15 psig and about 30 psig, equal to or between about 30 psig and about 60 psig, or equal to or between about 45 psig and about 60 psig.

In some examples the ultrafiltration and nanofiltration elements comprises spacers with thicknesses equal to or between about 30 mils and about 270 mils, with thicknesses equal to or between about 30 mils, 41 mils, 65 mils, 80 mils, 120 mils, 135 mils and 270 mils. In some examples the ultrafiltration element comprises spacers with thicknesses greater or equal to about 30 mils, greater or equal to about 45 mils, greater or equal to about 60 mils, greater or equal to about 75 mils, greater or equal to about 90 mils, greater or equal to about 105 mils, greater or equal to about 120 mils, or greater or equal to about 135 mils, or greater or equal to about 270 mils. In some examples the ultrafiltration element comprises spacers with thicknesses less than or equal to about 30 mils, less than or equal to about 45 mils, less than or equal to about 60 mils, less than or equal to about 75 mils, less than or equal to about 90 mils, less than or equal to about 105 mils, less than or equal to about 120 mils, or less than or equal to about 135 mils, or greater than or equal to about 270 mils.

In some examples at least about 90% of the insoluble particulates, typically greater than about 0.5 micron in diameter, remain in the retentate stream after the feed stream is subjected to the microporous membrane.

In some examples about 50% to about 70% of the high molecular weight solubles and fine particulates, typically greater than about 0.5 microns, are in the retentate stream from the microporous membrane.

In some examples the nanofiltration membrane is a low fouling spiral wound membrane comprised of one polysulfone, polyvinyl difluoride, polyethersulfone, polyacrylonitrile, polyetherimide, and the preferred membrane materials comprise polysulfone and polyvinyl difluoride.

In some examples the nominal pore size of the nanofiltration polymer membrane is less than about 20 nm, preferably less than about 15 nm.

In some examples the nanofiltration polymer membrane is a thin film composite membrane.

In some examples the thin film composite nanofiltration membrane comprises a microporous membrane substrate with pore size in the range of about 0.05 to about 0.2 microns and comprises a top nanoporous polymer layer of pore size less than about 20 nm or with a molecular weight cut-off less than about 5000.

In some examples the thin film composite nanofiltration membrane comprises a top nanoporous polymer layer produced by a dip-coating or interfacial polymerization process.

In some examples the nanofiltration membrane is formed directly using a phase inversion process.

In some examples the nanofiltration membrane provides a retentate comprising at least 80% of the lower molecular weight solubles, comprising lower molecular weight glutens.

In some examples the feed pressure to the microporous and nanofiltration membrane elements ranges from about 15 to about 200 psig.

In some examples the materials selected for the microporous and nanofiltration membranes are the same or different.

In some examples the temperature of overflow stream from the preprocess and the membrane steps of the first process are in the range of about 25° C. to about 65° C.

In some examples the prolamin recovery is about 1 to about 99% of the prolamin contained in the stream to the first process membrane unit.

In some examples the first microporous membrane has a MWCO in the range of about 2,000 to about 5,000, a range of about 1,000 to about 5,000, a range of about 2,000 to about 3,000, a range of about 3,000 to about 5,000, a range of about 1,000 to about 3,000, or a range of about 2,000 to about 4,000. In some examples the first microporous membrane has a MWCO less than about 5,000, less than about 4,000, less than about 3,000, less than about 2,000, or less than about 1,000. In some examples the second microporous membrane has a MWCO in the range of about 2,000 to about 5,000, a range of about 1,000 to about 5,000, a range of about 2,000 to about 3,000, a range of about 3,000 to about 5,000, a range of about 1,000 to about 3,000, or a range of about 2,000 to about 4,000. In some examples the second microporous membrane has a MWCO less than about 5,000, less than about 4,000, less than about 3,000, less than about 2,000, or less than about 1,000.

In some examples the membrane protein recovery system is integrated with a wet mill grain system comprising: (a) at least one gluten starch separator; (b) at least one gluten thickener; (c) at least one grain steeping vessel; and (d) at least two spiral wound membranes.

In some examples, at least one of the two membranes is in fluid contact with at least one gluten thickener; and at least one of the membranes is in fluid contact with the at least one grain steeping vessel.

In some examples the membrane protein recovery system and wet mill grain system comprise at least one microporous membrane in fluid contact with the at least one gluten thickener.

In some examples the membrane protein recovery system and wet mill grain system comprise at least one nanofiltration membrane in fluid contact with the at least one grain steeping vessel.

In some examples the membrane protein recovery system and wet mill grain system comprise a retentate stream from the at least one microporous membrane in fluid contact with the at least one gluten thickener.

In some examples the membrane protein recovery system and wet mill grain system comprise a retentate stream from the at least on ultrafiltration membrane in fluid contact with a separation device.

In some examples the membrane protein recovery system and wet mill grain system comprise a liquid stream from a separation device used to treat the retentate from the at least one microporous membrane in fluid contact with the at least one gluten thickener.

In some examples the membrane protein recovery system and wet mill grain system comprise a permeate stream from the at least one microporous membrane in fluid contact with the at least one nanofiltration membrane.

In some examples the membrane protein recovery system and wet mill grain system comprise a retentate stream from the at least one nanofiltration membrane in fluid contact with the at least one gluten thickener.

In some examples the membrane protein recovery system and wet mill grain system comprise a retentate stream from the at least on nanofiltration membrane in fluid contact with a separation device.

In some examples the membrane protein recovery system and wet mill grain system comprise a liquid stream from a separation device used to treat the retentate from the at least one nanofiltration membrane in fluid contact with the at least one gluten thickener.

In some examples the membrane protein recovery system and wet mill grain system comprise a permeate stream from the at least one nanofiltration membrane in fluid contact with the at least one steeping vessel.

In some examples the membrane protein recovery system and wet mill grain system comprise a permeate stream from the at least one nanofiltration membrane in fluid contact with a separation device, comprising a centrifuge, filter, decanter or another membrane element.

In some examples the membrane protein recovery system and wet mill grain system comprise a separation device, comprising a centrifuge, filter, decanter or another membrane element, in fluid contact with the grain steeping vessel.

In some examples the membrane protein recovery system and wet mill grain system comprise a permeate stream from the at least one nanofiltration membrane in fluid contact with a spray-drying device.

In some examples the membrane protein recovery system and wet mill grain system comprise a spray-drying device in fluid contact with the grain steeping vessel.

### Examples

The following examples are included to further describe some aspects of the present disclosure and should not be used to limit the scope of the disclosure.

### Membrane Preparation

In some examples, a microporous membrane was made from polyvinyldifluoride (PVDF) using a phase inversion method, by casting a polymer solution with PVDF, polyvinylpyrrolidone in N-methyl pyrrolidone. The PVDF membrane had a mean pore size of 0.1-0.2 micron. A nanofiltration membrane was made using an interfacial polymerization method, by firstly dip-coating an aqueous solution of piperazine and then followed by dip-coating a solution of trimesoyl chloride in isopar G to form a crosslinked polyamide layer on top of a microporous polysulfone membrane. The nanofiltration membrane had a molecular weight cut-off (MWCO) less than 5000.

### Membrane Stamp Test

In the first separation process (e.g., ultrafiltration), a PVDF membrane stamp of 47 mm in diameter was cut and mounted in a permeation system containing a stirred test cell. A feed solution with an insoluble solids level of about 4 wt % was introduced into the cell, and the permeate flux was measured at a feed pressure of 50 psig and a feed temperature of 23° C. The membrane stamp had a pressure and temperature normalized permeate flux of at least 2.6 LMH/bar. The permeate was a slightly yellow clear liquid. This ultrafiltration process may reject (in retentate) about 50% of soluble proteins in the feed.

In the second separation process, a nanofiltration membrane stamp of 47 mm in diameter was cut and mounted in a permeation system containing a stirred test cell. The permeate solution from the above ultrafiltration process was introduced into the cell, and the permeate flux was measured at a feed pressure of 50 psig and a feed temperature of 23° C. The membrane stamp had a pressure and temperature normalized permeate flux of 2.5 LMH/bar. The permeate was a colorless clear liquid. A protein rejection rate (in retentate) of 90% was achieved using this nanofiltration process. Upon standing, some white precipitate was found to develop in the NF permeate as well as in the UF permeate. Upon analysis using FTIR, the white precipitate was identified to be Corn Zein (prolamin).

In some examples, the zein solubility (prolamin solubility) may be dependent on the presence of other species, including glutens. In one example, once these other species were removed by the membrane processes of the present disclosure, a portion of the zein precipitated as a white powder, due to its limited solubility in aqueous streams. In some examples, dilution of the membrane process stream (e.g. the feed stream) with water can also result in precipitation of zein.

Some examples demonstrate the benefits of membrane fractionation using corn gluten rotary vacuum filter (RVF filtrate) and Merco gluten thickener overflow samples (MST) from a wet mill corn process. In a first ultrafiltration step, macro particles in the range of 0.5-10 microns were rejected (in the retentate), mostly high molecular weight glutens. In the second nanofiltration step low molecular weight glutens were rejected (in the retentate) and a stream comprising zein protein separated (from the permeate). Removal of the glutens resulted in precipitation of a portion of the corn zein, thus facilitating recovery of the zein.

One example protocol includes: providing gluten sources from 1) a gluten thickener overflow and 2) a rotary vacuum filter filtrate; receiving a liquid sample from the gluten sources, wherein the liquid sample is a suspension with yellow particulates settling at the bottom and a yellowish-clear solution on top; depositing the liquid sample in a 400 mL Amicon cell with applied N₂ pressure and magnetic stirring; removing yellow particulates with a coarse membrane (PE400); subjecting remaining solution to a polyethylene (PE) material, nanofiltration membrane; and collecting a clear permeate, wherein a white precipitate begins collecting after the membrane fractionation.

### Membrane Fabrication and Surface Modification

Further examples of membranes and membrane fabrication and surface modification are described herein.

Example 1: A sample (about 5×10 cm) of a PES ultrafiltration membrane (MWCO) approx. 50,000 g/mol is placed in a solution of one or more low molecular weight compounds (“modifying reagent”) in deionized water for 5 min dipped in, either benzoic acid, malonic acid, phenylphosphonic acid, 2-aminoethanesulfonic acid, toluenesulfonic acid. The sample is then placed on a glass plate and irradiated in the electron accelerator. The membrane is then rinsed 3 times with deionized water and dried at 100° C. All resulting membranes show reduction in nonspecific protein adsorption.

Example 2: A sample (about 5×10 cm) of a PES microfiltration membrane (mean pore size about 0.4 microns) is immersed for 5 min in a solution of low molecular weight compounds such as but not limited to glycerin, ethylamine, triethylamine, methylmalonic acid, naphtalenedisulfonic acid, taurine in deionized water. The sample is then placed on a glass plate and irradiated in the electron accelerator. The membrane is then rinsed 3 times with deionized water and dried at 100° C. All resulting membranes show reduction in nonspecific protein adsorption.

Example 3: A sample (Ø 47 mm) of a PES microfiltration membrane with an average pore size of 0.2 micron is immersed for 5 min in a solution of 1 wt % phosphorylcholine in deionized water. The sample is then placed on a glass plate and irradiated after removal of the surface water in the electron accelerator at a dose of 200 kGy. The membrane is then incubated for 7 days in a Soxhlett Apparatus continuously extracted with boiling water and dried at 100° C. The seven-day continuous extraction with boiling water in a Soxhlett apparatus demonstrated the stable modification on the inner membrane surface. Resulting membrane shows reduction in nonspecific protein adsorption.

Example 4: A sample (about 5×10 cm) of a PES ultrafiltration membrane (MWCO about 50,000 g/mol) is immersed for 5 min in a solution of low molecular weight of diethyl phosphoramidate, phosphonopropionic acid, glutamine, glucose and phosphorylcholine in deionized water. The sample is then placed on a glass plate and irradiated in the electron accelerator. The membrane is then rinsed 3 times with deionized water and dried at 100° C. All resulting membranes show reduction in nonspecific protein adsorption.

Example 5: A sample (about 20×10 cm) of a PES ultrafiltration membrane (MWCO about 50,000 g/mol) is immersed for 5 min in a solution of 30 wt % phenylphosphonic acid in deionized water. Subsequently, the sample is placed on a glass plate and irradiated in the electron accelerator with a dose of 100 kGy. The membrane is then rinsed 3 times with deionized water and dried at 100° C. The resulting properties shows clearly that all molecules show a reduction in non-specific protein adsorption. To check the modification, a cation exchange was additionally carried out. For this purpose, the phenylphosphonic acid-modified and unmodified membrane samples (10 disks each, Ø 10 mm) were immersed for comparison in a SrCl₂ solution (5% in deionized water) for 30 minutes. Subsequently, the membrane was rinsed with deionized water and dried at 100° C. for 30 min. Strontium may subsequently be detected by an inductively coupled plasma optical emission spectroscopy (ICP-OES) device and thus concluded that phosphonic acid groups were present on the membrane. Thus, it may be shown that membrane modification with phenylphosphonic acid led to a 22% increase in Sr uptake, which, in combination with the found P values, also indicates the presence of additional phosphonic acid groups on the membrane.

Example 6: A sample (about 5×10 cm) of a PAN ultrafiltration membrane (MWCO about 20,000 g/mol) is immersed for 5 min in a solution of low molecular weight compounds such as phenylphosphonic acid and toluene sulfonic acid in deionized water. The sample is then placed on a glass plate and irradiated in the electron accelerator. The membrane is then rinsed 3 times with deionized water and dried at 75° C.

Example 7: A sample (Ø 47 mm) of a PSf microfiltration membrane with an average pore size of 0.2 μm is immersed for 5 min in a solution of low molecular weight compounds such as glutamin and phenylphosphonic acid in deionized water. The sample is then placed on a glass plate and irradiated in the electron accelerator. The membrane is then rinsed 3 times with deionized water and dried at 100° C. The resulting properties of the membrane show reduction in nonspecific protein adsorption.

### Preparation of a Charged Membrane

Sulfonation Reaction: 10.35 g of polyethersulfone (PES) (BASF Ultrason E) was placed in a stoppered 125 mL Erlenmeyer flask containing 70 g of anhydrous sulfolane with a magnetic stir bar. This flask was heated at 130° C. in a glycerin bath on top of a magnetic stirring hotplate for 1 hour, at which point all of the PES was dissolved. 4.8 mL of fuming sulfuric acid (Alfa Aesar 18-24% free SO₃) was added into this solution over approximately 4 minutes at a temperature range of 127-134° C. The reaction solution was stirred further for 1 hour while maintaining this temperature. This clear, brownish-colored solution was removed from the heating bath and allowed to cool at room temperature. A sample of this solution was precipitated in deionized water and washed several times, then dried 3%2 hours at 105-110° C. The final sulfonated product (SPES) had an ion exchange capacity (IEC) of 0.97 milli-equivalent per gram (meq/g).

Casting solution: 81.5 g PES was dissolved in 183 g of N-methyl-2-pyrrolidinone (NMP) as one of the components of the casting solution. To this was added directly 39.8 g of the above sulfonation reaction solution, 194.4 g sulfolane, and 1.31 g concentrated sulfuric acid, to give a final 500 g casting solution formulation consisting of 16.3% PES, 1% SPES, 36.6% NMP, 45.1% Sulfolane, and 1% H₂SO₄.

Alternatively, a method comprising the steps of: 1) sulfonating a first poly(aryl ether) to provide a sulfonated poly(aryl ether); 2) combining the sulfonated poly(aryl ether) and a second poly(aryl ether) to provide a casting solution; 3) casting the solution to provide a filtration membrane, wherein the sulfonated poly(aryl ether) is not isolated prior to casting the membrane.

Example casting solution was cast on the polyester and/or polypropylene/polyethylene blend backing fabric using phase inversion method in none-solvent.

Casting solutions following the composition presented in Table 1.

TABLE 1
Polysulfone, polyethersulfone, sulfonated polysulfone
and sulfonated polyethersulfone casting solutions

| | | | | | | | | Sulfuric |
|---|---|---|---|---|---|---|---|---|
| | PES | SPS | IEC | SPES | IEC | NMP | Sulfolane | acid |
| No. | wt % | wt % | meq/g | wt % | meq/g | wt % | Wt % | wt % |
| 1 | 21.5 | 0.90 | 2.3 | — | — | 22.4 | 55.2 | — |
| 2 | 21.5 | 0.45 | 2.3 | — | — | 22.6 | 55.5 | — |
| 3 | 22.5 | 0.90 | 2.3 | — | — | 22.1 | 54.5 | — |
| 4 | 21.5 | 1.34 | 2.3 | — | — | 22.3 | 54.9 | — |
| 5 | 18.3 | 0.90 | 2.3 | — | — | 36.2 | 44.6 | — |
| 6 | 19.3 | 0.90 | 1.8 | — | — | 35.6 | 43.7 | — |
| 7 | 19.3 | 0.90 | 0.7 | — | — | 35.6 | 43.7 | — |
| 8 | 19.3 | — | — | 0.90 | 0.90 | 35.6 | 43.7 | — |
| 9 | 19.3 | — | — | 0.90 | 2.0 | 35.6 | 43.7 | — |
| 10 | 19.3 | — | — | 0.90 | 3.9 | 35.6 | 43.7 | — |
| 11 | 20.5 | — | — | 2.00 | 3.9 | 34.8 | 42.7 | — |
| 12 | 21.5 | — | — | 1.00 | 3.9 | 34.8 | 42.7 | — |
| 13 | 20.5 | — | — | 2.00 | 3.0 | 34.8 | 42.7 | — |
| 14 | 17.3 | — | — | 1.45 | 0.9 | 36.2 | 44.5 | — |
| 15 | 17.4 | — | — | 2.33 | 2.1 | 35.9 | 44.2 | — |
| 16 | 18.4 | — | — | 0.90 | 0.90 | 36.2 | 44.5 | — |
| 17 | 18.3 | — | — | 0.90 | 1.4 | 36.2 | 44.6 | — |
| 18 | 18.3 | — | — | 0.90 | 0.80 | 36.2 | 44.6 | — |
| 19 | 19.9 | — | — | 0.83 | 1.2 | 36.2 | 53.1 | — |
| 20 | 16.3 | — | — | 2.00 | 0.90 | 36.2 | 44.5 | 1.0 |
| 21 | 16.3 | — | — | 1.00 | 0.90 | 36.6 | 45.1 | 1.0 |
| 22 | 15.3 | — | — | 2.00 | 0.90 | 36.6 | 45.1 | 1.0 |
| 23 | 13.7 | — | — | 4.00 | 1.0 | 36.2 | 44.5 | 1.0 |
| 24 | 22.3 | — | — | 1.20 | 1.0 | 35.0 | — | 1.0 |
| 25 | 21.6 | — | — | 2.40 | 1.0 | 35.0 | — | 1.0 |
| 26 | 18.0 | — | — | 6.00 | 1.0 | 35.0 | — | 1.0 |
| 27 | 12.0 | — | — | 12.0 | 1.0 | 35.0 | — | 1.0 |
| 28 | 0 | — | — | 24.0 | 1.0 | 35.0 | — | 1.0 |

Recovering of the Protein from Soy and Pea Whey

This example demonstrates the fractionation of pea proteins, such as Glycinin, Albumin, Legumin, Vicilin, Glycinin, β-conglycinin as shown in Table 2. As demonstrated in the table, these specific proteins may be selectively rejected at a high efficiency as well as further concentrated for further processing.

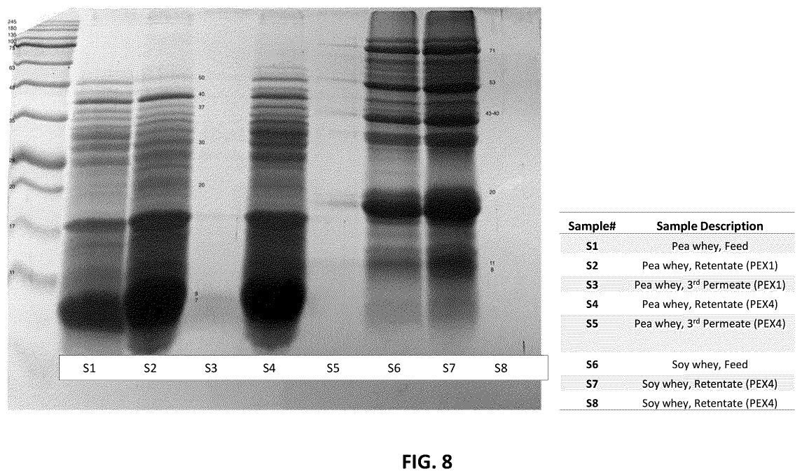

Protein content was determined using the combustion method with a Protein/Nitrogen Analyzer. Protein fractionation (SDS-PAGE) was done to quantify the relative proportions of legumin and vicilin (LN) for the pea samples, and glycinin/beta-conglycinin (GM/B) for the soy samples, and albumin and globulin (A/G) for all samples (FIG. 8). The protein bands were measured via volume, where volume is determined by the sum of pixel intensity for all pixels in each section. Legumin/Vicilin, Glycinin.

TABLE 2
Fractionation of Proteins

| | | Protein | | |
|---|---|---|---|---|
| | | Content | Legumin (L)/ | Albumin (A)/ |
| Sample# | Sample Description | (%) | Vicilin (V) | Globulin(G) |
| S1 | Pea whey, Feed | 0.69 | 1.07 ± 0.12 | 1.18 ± 0.09 |
| S2 | Pea whey, Retentate | 2.04 | 0.76 ± 0.01 | 1.02 ± 0.38 |
| | (PEX1) | | | |
| S3 | Pea whey, 3^{rd} | 0.32 | ND | ND |
| | Permeate (PEX1) | | | |
| S4 | Pea whey, Retentate | 1.63 | 1.04 ± 0.05 | 1.75 ± 0.23 |
| | (PEX4) | | | |
| S5 | Pea whey, 3^{rd} | 0.33 | ND | ND |
| | Permeate (PEX4) | | | |
| | | | Glycinin (GL)/ | |
| | | | β-conglycinin | Albumin (A)/ |
| | | | (BC) | Globulin (G) |
| S6 | Soy whey, Feed | 0.86 | 2.66 ± 0.08 | 0.55 ± 0.09 |
| S7 | Soy whey, Retentate | 2.75 | 3.21 ± 0.00 | 0.67 ± 0.13 |
| | (PEX4) | | | |
| S8 | Soy whey, Retentate | 0.30 | ND | ND |
| | (PEX4) | | | |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-133. (canceled)

134. A method of recovering fine particulate, or protein species from a feed from a wet mill grain process, the method comprising:
(a) subjecting the feed from the wet mill grain process to a first separation process utilizing a first membrane under conditions sufficient to provide a first retentate stream and a first permeate stream,
wherein the first retentate stream comprises one or more higher molecular weight proteins having an average molecular weight of greater than about 75 kilodalton, and
wherein the first permeate stream comprises one or more lower molecular weight proteins having an average molecular weight of less than or equal to about 75 kilodalton; and
(b) subjecting the first permeate stream to a second separation process utilizing a second membrane under conditions sufficient to provide a second retentate stream and a second permeate stream,
wherein the second retentate stream comprises glutens and
wherein the second permeate stream comprises prolamins.

135. The method of claim 134, wherein the first membrane, comprising a microporous membrane or an ultrafiltration membrane, has a nominal pore size of about 0.02 micrometers to about 0.50 micrometers.

136. The method of claim 134, wherein the second membrane comprises a nanofiltration membrane having a nominal pore size of less than about 20 nanometers.

137. The method of claim 134, wherein the one or more lower molecular weight proteins comprise glutens or prolamins.

138. The method of claim 134, further comprising utilizing at least one spiral wound membrane element as an additional separation process to further filter the prolamins from the second retentate stream.

139. The method of claim 134, further comprising directing the second permeate stream to pass through a nanofiltration membrane unit to recover protein species comprising prolamins.

140. The method of claim 134, wherein recovery of a stream comprising prolamins is increased by removal of a stream comprising glutens.

141. The method of claim 134, further comprising recovering one or more protein species or non-protein species from at least one of (i) the first retentate stream, (ii) the first permeate stream, (iii) the second retentate stream, and (iv) the second permeate stream,
wherein the one or more protein species comprises glutelins, prolamins or glutens, and
wherein the non-protein species comprise carbohydrates, starches, enzymes, alcohols, aldehydes, fats or other low molecular weight organic species.

142. The method of claim 134, wherein the feed from the wet mill grain process to the first separation process is supplied from an overflow of a mechanical separation device or gravity separation device.

143. The method of claim 134, wherein the feed from the wet mill grain process is an aqueous stream with no added organic solvent.

144. The method of claim 134, wherein the first membrane comprises a low fouling spiral wound membrane comprising at least one material selected from the group consisting of polysulfone, polyvinyl difluoride, polyethersulfone, polyacrylonitrile, and polyetherimide.

145. The method of claim 136, wherein the nanofiltration membrane is a low fouling spiral wound membrane, wherein the nanofiltration membrane comprises (i) a microporous substrate comprising at least one member selected from the group consisting of polysulfone, polyvinyl difluoride, polyethersulfone, polyacrylonitriles, and polyetherimide material and (ii) a top interfacial coating or separation layer comprising pores with pore sizes ranging from 5 nm to about 100 nm, wherein the top interfacial coating or separation layer comprises interfacial crosslinked polyamide layer.

146. The method of claim 134, wherein the spiral wound membrane elements comprise a spacer element with a thickness equal to or between about 30 mils and about 270 mils.

147. The method of claim 134, wherein the first separation process or the second separation process is operated at a feed pressure of about 15 pounds per square inch gauge to about 200 pounds per square inch gauge.

148. The method of claim 134, wherein the first retentate stream comprises at least about 90% of the insoluble particulates greater than about 0.5 micrometers in diameter.

149. The method of claim 134, wherein a reject stream from the first membrane comprises about 5% to about 70% of high molecular weight solubles and fine particulates greater than 0.5 microns in diameter.

150. The method of claim 145, wherein the nanofiltration membrane removes at least 80% of the low molecular weight solubles in the first permeate stream.

151. The method of claim 141, wherein a recovery stream comprises greater than 10% of low molecular weight proteins as originally comprised in the feed from the wet mill grain process.

152. The method of claim 141, wherein a recovery stream comprises about 1% to about 99% of the prolamin contained in the feed from the wet mill grain process.

153. The method of claim 134, wherein one or both of the first membrane and the second membrane comprises a charged membrane, wherein the charged membrane is characterized by a net negative charge or comprises one or more functional groups along a surface of the charged membrane.
